(19) United States
(12) Patent Application Publication
(10) Pub. No.: US 2006/0293340 A1
(43) Pub. Date: Dec. 28, 2006

(54) 2-Aminopyrimidine derivatives as raf kinase inhibitors

(75) Inventors: David Bryant Batt, Wayland, MA (US); Timothy Michael Ramsey, Weston, MA (US); Michael Lloyd Sabio, Ridgewood, NJ (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 10/560,352

(22) PCT Filed: Jun. 11, 2004
(86) PCT No.: PCT/EP04/06317

§ 371 Date: May 12, 2006

Related U.S. Application Data
(60) Provisional application No. 60/478,709, filed on Jun. 13, 2003.

(51) Int. Cl. A61K 31/506; C07D 403/02

(52) U.S. Cl. 514/255.05; 514/275; 544/295; 544/296; 544/330; 544/331

(57) ABSTRACT

This application discloses compounds that inhibit Raf kinase having the formula (I), wherein R₁ is a phenyl radical or a heteroaryl radical; and R₂ is a phenyl radical; or an N-oxide or a pharmaceutically acceptable salt thereof. The compounds are useful for the treatment of proliferative diseases, such as cancer.

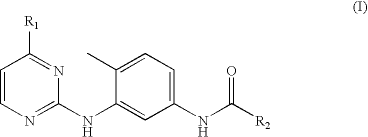

### SUMMARY

The present invention relates to the discovery that certain compounds inhibit RAF kinase, a serine/threonine kinase that functions in the MAP kinase signaling pathway, and to the use of the compounds for the treatment of diseases characterized by excessive signaling in the MAP kinase signaling pathway, for example proliferative diseases like certain cancers.

### BACKGROUND

Cells communicate various aspects of their extracellular environment to the nucleus by using various signal transducton pathways. Many of these signals are transmitted by protein kinases which activate various factors through the transfer of phosphate groups. Disruption of signal transduction by inhibiting appropriate kinase activity can have a clinical benefit as has been demonstrated by imatinib, an inhibitor of bcr-abl kinase, which is marketed as its mesylate salt under the brand GLEEVEC (in the United States) or GLIVEC.

The MAP kinase signaling pathway is known in the art as one of the pathways for growth factors to send their signal to proliferate from the extracellular environment to the cell nucleus. The growth factors activate transmembrane receptors located on the cell surface which in turn start a cascade whereby RAS is activated and recruits RAF kinase to the membrane where it is activated and in turn activates MEK kinase which then activates ERK kinase. Activated ERK kinase can move to the nucleus where it activates various gene transcription factors. Aberrations in this pathway can lead to altered gene transcription, cellular growth and contribute to tumorogenicity by negatively regulating apoptosis and transmitting proliferative and angiogenic signals. Inhibitors of RAF kinase have been shown to block signaling through the MAP kinase signaling pathway.

The RAF kinase family is known to have three members designated C-RAF, also known as RAF-1, B-RAF and A-RAF. It has been reported that B-RAF kinase is commonly activated by one of several somatic point mutations in human cancer, including 59% of the melanoma cell lines tested. See, Davies, H. et al, Nature 417, 949-954 (2002). This invention relates to the discovery of a class of compounds that efficiently inhibit one or more members of the RAF kinase family.

The RAF kinase inhibiting property of the compounds makes them useful as therapeutic agents for the treatment for proliferative diseases characterized by an aberrant MAP kinase signaling pathway, particularly many cancers characterized by overexpression of RAF kinase or an activating mutation of RAF kinase, such as melanoma having mutated B-RAF, especially wherein the mutated B-RAF is the V599E mutant. The present invention also provides a method of treating other conditions characterized by an aberrant MAP kinase signaling pathway, particularly where B-RAF is mutated, for example benign Nevi moles having mutated B-RAF, with the compounds.

### DESCRIPTION

The present invention relates to compounds of formula (I) and to a method of treating a patient having a disease characterized by excessive signaling through the MAP kinase pathway, which comprises administering to the patient an effective RAF kinase inhibiting amount of a compound of formula (I).

The inventive compounds are described by formula (I)

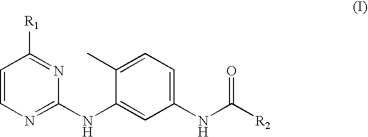

wherein

R₁ is a phenyl radical or a heteroaryl radical; and

R₂ is a phenyl radical;

or an N-oxide or a pharmaceutically acceptable salt thereof.

Within the context of the present disclosure, the general terms used herein to describe compounds of formula (I) have the following meanings, unless indicated otherwise.

The term “lower” when referring to substituents such as alkyl, alkoxy, alkyl amine, alkylthio and the like denotes a radical having up to and including a maximum of 7, especially from 1 up to and including a maximum of 4, carbon atoms, the radicals in question being unbranched or branched one or more times.

The alkyl portion of lower alkyl, lower alkoxy, mono- or di-lower alkyl amino, lower alkyl thio and other substituents with an alkyl portion is especially C₁-C₄alkyl, for example n-butyl, sec-butyl, tert-butyl, n-propyl, isopropyl, methyl or ethyl. Such alkyl substituents are unsubstituted or substituted by halogen, hydroxy, nitro, cyano, lower alkoxy, C₃-C₇ cycloalkyl, amino, or mono- or di-lower alkyl amino, unless otherwise indicated.

Halo-lower alkyl, halo-lower alkyloxy, halo-lower alkylthio and the like refer to substituents having an alkyl portion wherein the alkyl portion is mono- to completely substituted by halogen. Halo-lower alkyl, halo-lower alkyloxy, halo-lower alkylthio and the like are included within substituted lower alkyl, substituted lower alkoxy, substituted lower alkylthio and the like.

Halogen is especially fluorine, chlorine, bromine or iodine, more especially fluorine, chlorine or bromine, in particular fluorine.

Any reference to compounds, salts and the like in the plural is always to be understood as including one compound, one salt or the like.

A phenyl radical is generally an unsubstituted phenyl or phenyl that is substituted with from 1-5, preferably 1 or 2, substituents. Appropriate substituents include, but are not limited to, amino, mono- or di-lower alkyl substituted amino, wherein the lower alkyl substitents may be unsubstituted or further substituted by those substitutents listed above for alkyl groups, halogen, lower alkyl, substituted lower alkyl, hydroxy, lower alkoxy, substituted lower alkoxy, nitro, cyano, mercapto, lower alkylthio, halo-lower alkylthio, heterocyclyl, heteroaryl, heterocyclylalkyl, heteroarylalkyl, lower alkanoyl, carbamoyl, and N-mono- or N,N-di-lower alkyl substituted carbamoyl, wherein the lower alkyl substitents may be unsubstituted or further substituted.

R₁ as a phenyl radical is especially unsubstituted phenyl or phenyl which is substituted by one or more substituents, preferably up to three, especially one substituent. Especially important substituents for R₁ phenyl radicals include amino, mono- or di-lower alkyl amino, where the alkyl groups are unsubstituted or substituted, halogen, lower alkyl, substituted lower alkyl, hydroxy, lower alkoxy, substituted lower alkoxy, nitro, cyano, mercapto, lower alkylthio and substituted lower alkylthio.

R₁ as a phenyl radical is especially phenyl that is unsubstituted or substituted by one or two identical or different substituents selected from halogen, especially fluorine or chlorine; lower alkyl, especially methyl, ethyl, propyl or t-butyl; halo-lower alkyl, especially trifluoromethyl; hydroxy; lower alkoxy, especially methoxy or ethoxy; halo-lower alkyloxy, such as trifluoromethoxy or 1,1,2,2-tetrafluoroethyloxy; more especially by one substituent selected from unsubstituted or substituted lower alkyl, especially methyl, halo-lower alkyl, such as trifluoromethyl, unsubstituted or substituted lower alkoxy, especially methoxy and halo-lower alkoxy, especially trifluoromethoxy.

In one embodiment, the substituent is at the position which is meta or para to the bond to the pyrimidine ring.

Important R₁ phenyl radicals include unsubstituted phenyl and lower alkoxy-substituted phenyl, especially wherein the lower alkoxy substituent is at the position meta or para to the bond to the pyrimidine ring.

R₂ as a phenyl radical is generally a phenyl ring that is unsubstituted or preferably substituted by one or more substituents, preferably up to three, especially one or two substituents. The substituents are especially amino, mono- or di-lower alkyl-substituted amino, wherein the alkyl groups are unsubstituted or further substituted, especially by halogen or lower alkoxy; -halogen, unsubstituted or substituted lower alkyl, unsubstituted or substituted lower alkoxy, hydroxy, nitro, cyano, lower alkanoyl, carbamoyl, N-mono- or N,N-di-lower alkyl substituted carbamoyl, mercapto, lower alkylthio and halo-lower alkylthio.

R₂ as a phenyl radical is preferably phenyl that is substituted by one or two identical or different substituents selected from halogen, especially fluorine or chlorine; mono- or di-lower alkyl-substituted amino, especially dimethylamino or diethylamino; lower alkyl, especially methyl or ethyl; halo-lower alkyl, especially difluoromethyl, trifluoromethyl, 2,2,2-trifluoroethyl, or 1,1,2,2-tetrafluoroethyl; lower alkoxy, especially methoxy or ethoxy; halo-lower alkoxy, especially difluoromethoxy, trifluoromethoxy, 2,2,2-trifluoroethoxy, or 1,1,2,2-tetrafluoroethoxy, lower alkylthio, such as methylmercapto, halo-lower alkylthio, such as difluoromethylthio, trifluoromethylthio, 2,2,2-trifluoroethylthio, or 1,1,2,2-tetrafluoroethylthio. R₂ as a phenyl radical is more especially phenyl substituted by one or two identical or different substituents selected from unsubstituted or halo-substituted lower alkyl, unsubstituted or halo-substituted lower alkoxy, unsubstituted or halo-substituted lower alkylthio and halogen, especially fluorine or chlorine.

R₂ is especially trifluoromethylphenyl, particularly 3-trifluoromethylphenyl.

Preferably, an R₂ phenyl radical will be substituted in the position meta to the linking aminocarbonyl group.

A heteroaryl radical is especially a 5 to 7 membered aromatic ring comprising from 1 to 3 heteroatoms selected from N, O and S. The heteroaryl radical is unsubstituted or substituted by one or more, especially from one to three, for example one, identical or different substituents. Important substituents on heteroaryl radicals are those selected from the group consisting of halogen, for example, fluorine or chlorine; mono- or di-lower alkyl-substituted amino wherein the alkyl groups are unsubstituted or substituted by halogen, hydroxy, nitro, cyano, lower alkoxy, C₃-C₇ cycloalkyl, a heterocyclic radical or a heteroaryl radical; lower alkyl, such as methyl or ethyl; halo-lower alkyl, such as trifluoromethyl; lower alkoxy, such as methoxy or ethoxy; halo-lower alkoxy, for example, trifluoromethoxy; lower alkylthio, such as methylmercapto, halo-lower alkylthio, such as trifluoromethylthio, a heteroaryl radical, heteroaryl-lower-alkylene, a heterocyclic radical or heterocyclic-lower-alkylene.

Heteroaryl-lower-alkylene and heterocyclic-lower-alkylene are substituents of the formula het-C₁-C₄-alkylene- where het is a heteroaryl or heterocyclic radical.

R₁ as a heteroaryl radical is especially a thiazolyl, pyrazinyl, pyrimidinyl or pyridyl radical, especially a 2-thiazolyl, 2-pyrazinyl, 5-pyrimidinyl or 3-pyridyl radical.

Preferably, R₁ as a heteroaryl radical is a pyridyl radical, especially a 3-pyridyl radical, more particularly a 6-substituted-3-pyridyl radical.

A heterocyclic radical is especially a five- or six-membered non-aromatic ring having 1 or 2 hetero atoms selected from the group consisting of nitrogen, oxygen and sulfur, which heterocycle may be fully or partially saturated, and is unsubstituted or substituted, especially by unsubstituted or substituted lower alkyl. Heterocyclic radicals include morphilino, thiomorphilino, piperidinyl, piperazinyl, and the like.

Salts are especially the pharmaceutically acceptable acid addition salts of compounds of formula I. Such salts are formed, for example, by compounds of formula I having a basic nitrogen atom as acid addition salts, preferably with organic or inorganic acids, especially the pharmaceutically acceptable salts. Suitable inorganic acids are, for example, hydrohalic acids, such as hydrochloric acid; sulfuric acid; or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, 2-hydroxybutyric acid, gluconic acid, glucosemonocarboxylic acid, fumaric acid, succinic acid, adipic acid, pimelic acid, suberic acid, azelaic acid, malic acid, tartaric acid, citric acid, glucaric acid, galactaric acid, amino acids, such as glutamic acid, aspartic acid, N-methylglycine, acetylaminoacetic acid, N-acetylasparagine, N-acetylcysteine, pyruvic acid, acetoacetic acid, phosphoserine, 2- or 3-glycerophosphoric acid, maleic acid, hydroxymaleic acid, methylmaleic acid, cyclohexanecarboxylic acid, benzoic acid, salicylic acid, 1- or 3-hydroxynaphthyl-2-carboxylic acid, 3,4,5-trimethoxybenzoic acid, 2-phenoxybenzoic acid, 2-acetoxybenzoic acid, 4-aminosalicylic acid, phthalic acid, phenylacetic acid, glucuronic acid, galacturonic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalenedisulfonic acid, N-cyclohexylsulfamic acid, N-methyl-, N-ethyl- or N-propyl-sulfamic acid, or other organic protonic acids% such as ascorbic acid.

For isolation or purification it is also possible to use pharmaceutically unacceptable salts, for example picrates or perchlorates. Only the pharmaceutically acceptable salts or the free compounds (optionally in the form of pharmaceutical compositions) are used therapeutically, and those are therefore preferred.

In view of the close relationship between the novel compounds in free form and in the form of their salts, including also those salts which can be used as intermediates, for example in the purification of the novel compounds or for their identification, hereinbefore and hereinafter any reference to the free compounds is also to be understood as including the corresponding salts, as appropriate and expedient.

An important embodiment of the compounds of the formula I are those compounds wherein R₁ is selected from a phenyl radical, a thiazolyl radical, a pyrazinyl radical, a pyrimidinyl radical or a pyridyl radical; especially those wherein R₂ is halo-phenyl (phenyl substituted by halogen), halo-lower alkylphenyl, halo-lower alkoxyphenyl or halo-lower alkylthiophenyl; most particularly those wherein the alkyl portion of halo-lower alkylphenyl, halo-lower alkoxyphenyl or halo-lower alkylthiophenyl substituents is from mono-substituted to entirely substituted by fluorine, such as 2-fluoroethyl, trifluoromethyl or pentafluoroethyl.

An important embodiment of the present invention are the compounds of formula II

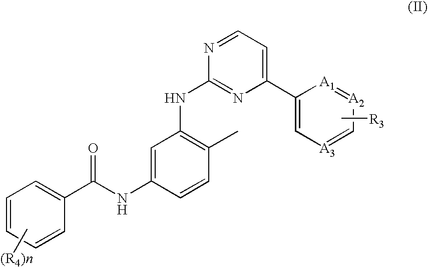

wherein

n is 0, 1 or2;

A₁, A₂ and A₃ are C, or A₁ and A₂ are C and A₃ is N, or A₁ and A₃ are N and A₂ is C, or A₁ is C and A₂ and A₃ are N;

R₃ is —NR₅R₆, halogen, —O—R₈, —S—R₈, or lower alkyl which is unsubstituted or substituted by halogen, hydroxy, lower alkoxy, —NR₇R₈, or a heteroaryl or heterocyclic radical attached at a ring carbon;

R₄ is amino, mono- or di-lower alkyl-substituted amino, wherein the alkyl groups are unsubstituted or substituted by halogen or lower alkoxy; halogen, lower alkyl, halo-lower alkyl, lower alkoxy, halo-lower alkoxy, hydroxy, lower alkanoyl, carbamoyl, N-mono- or N,N-di-substituted carbamoyl, mercapto, lower alkylthio or halo-lower alkylthio;

R₅, R₆, R₇ and R₈ are independently hydrogen, a heteroaryl or heterocyclic radical attached at a ring carbon, lower alkyl, C₃-C₈cycloalkyl, C₃-C₈cycloalkyl-lower alkylene, lower alkyl which is substituted by hydroxy, lower alkoxy, a heteroaryl radical, a heterocyclic radical, amino, mono- or di-lower alkyl amino or R₅ and R₆ and/or R₇ and R₈ together with the nitrogen form a heteroaromatic or heterocyclic radical;

R₈ is a heterocyclic radical, a heteroaromatic radical, heteroaryl-lower-alkylene, heterocyclic-lower-alkylene, lower alkyl or lower alkyl which is substituted by hydroxy, lower alkoxy or —NR₇R₈;

or an N-oxide or a pharmaceutically acceptable salt thereof.

When R₃ is lower alkyl substituted by a heteroaryl or heterocyclic radical attached at a ring carbon, the lower alkyl is bonded to a ring carbon of the heteroaryl or heterocyclic radical, for example, 3-(4-pyridyl)propyl or 4-piperidinylmethyl.

When R₅—R₈ is a heteroaryl or heterocyclic radical attached at a ring carbon, the amino nitrogen is directly bonded to a ring carbon of the heteroaryl or heterocyclic radical, for example when —NR₅R₆ is 4-pyridylamino or 4-piperidinylamino.

At least one R₄ substituent is preferably located at the position meta to the carbonyl.

The R₄ substituents are especially selected from halogen, especially fluorine or chlorine; most particularly fluorine, mono- or di-lower alkyl-substituted amino, especially dimethylamino or diethylamino; lower alkyl, especially methyl or ethyl; halo-lower alkyl, especially difluoromethyl, trifluoromethyl, 2,2,2-trifluoroethyl, or 1,1,2,2-tetrafluoroethyl; lower alkoxy, especially methoxy or ethoxy; halo-lower alkoxy, especially difluoromethoxy, trifluoromethoxy, 2,2,2-trifluoroethoxy, and 1,1,2,2-tetrafluoroethoxy, lower alkylthio, such as methylmercapto, halo-lower alkylthio, such as difluoromethylthio, trifluoromethylthio, 2,2,2-trifluoroethylthio, or 1,1,2,2-tetrafluoroethylthio.

R₄ is especially one or two identical or different substituents selected from unsubstituted or halo-substituted lower alkyl, unsubstituted or halo-substituted lower alkoxy, unsubstituted or halo-substituted lower alkylthio and halogen, especially fluorine or chlorine.

A preferred embodiment of the present invention are the compounds of formula (III)

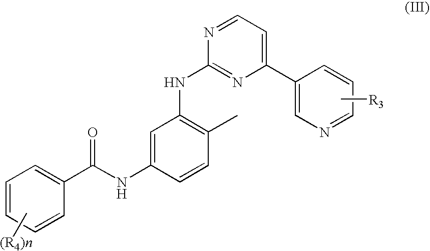

wherein

n is 0, 1 or 2;

R₃ is —NR₅R₆, halogen, —O—R₈, —S—R₈, or lower alkyl which is unsubstituted or substituted by halogen, hydroxy, lower alkoxy, —NR₇R₈, or a heteroaryl or heterocyclic radical attached at a ring carbon;

R₄ is amino, mono- or di-lower alkyl-substituted amino, wherein the alkyl groups are unsubstituted or substituted by halogen or lower alkoxy; halogen, lower alkyl, halo-lower alkyl, lower alkoxy, halo-lower alkoxy, hydroxy, lower alkanoyl, carbamoyl, N-mono- or N,N-di-substituted carbamoyl, mercapto, lower alkylthio or halo-lower alkylthio;

R₅, R₆, R₇ and R₈ are independently hydrogen, a heteroaryl or heterocyclic radical attached at a ring carbon, lower alkyl, C₃-C₈cycloalkyl, C₃-C₈cycloalkyl-lower alkylene, lower alkyl which is substituted by hydroxy, lower alkoxy, a heteroaryl radical, a heterocyclic radical, amino, mono- or di-lower alkyl amino or R₅ and R₆ or R₇ and R₈ together with the nitrogen form a heteroaromatic or heterocyclic radical;

R₈ is a heterocyclic radical, a heteroaromatic radical, heteroaryl-lower-alkylene, heterocyclic-lower-alkylene, lower alkyl or lower alkyl which is substituted by hydroxy, lower alkoxy or —NR₇R₈;

or an N-oxide or a pharmaceutically acceptable salt thereof.

Among the compounds of formula III, important embodiments Include those compounds wherein R₄ is halo-lower alkyl, for example, trifluoromethyl, halo-lower alkoxy, for example, trifluoromethoxy, or halo-lower alkylthio, for example, trifluoromethylthio, especially those wherein one R₄ substitutent is located at the 3-position (meta) relative to the bond to the carbonyl. In an especially important embodiment, the R₄ substitutent is selected from 3-trifluoromethyl, 3-trifluoromethoxy and 3-trifluoromethylthio, especially 3-trifluoromethyl.

Further important embodiments of the compounds of formula III include those compounds wherein R₃ is —NR₅R₆ and one of R₅ and R₆ is lower alkyl substituted by —NR₇R₈ and R₇ and R₈ together with the nitrogen form a heteroaromatic or heterocyclic radical; especially wherein the heteroaromatic or heterocyclic radical is selected from morphilino, thiomorphilino, piperazinyl, piperidinyl, and pyridyl.

Further important embodiments of the compounds of formula III include those compounds wherein —NR₅R₆ is a heteroaryl or heterocyclic radical, especially piperazinyl, 4-methylpiperazinyl, piperidinyl, 4-hydroxypiperidinyl, morphilino and thiomorphilino.

Further important embodiments of the compounds of formula III include those compounds wherein R₈ is lower alkyl or lower alkyl substituted by hydroxy or lower alkoxy, especially methoxy or ethoxy; for example those compounds wherein R₃ is 2-methoxyethoxy, 3-methyoxypropyloxy, 3-ethoxypropyloxy, 3-methoxypropylthio or 2-hydroxypropylthio.

Further important embodiments of the compounds of formula III include those compounds wherein R₈ is a heteroaryl or heterocyclic radical, such as those compounds wherein R₃ is 3-pyridyloxy or 1-methyl-piperidin-4-yloxy.

In a particularly preferred embodiment of the compounds of formula III, the R₃ substitutent is located at the 6-position of the pyridine ring to which it is attached. Thus, the present invention especially includes compounds of the formula (IIa)

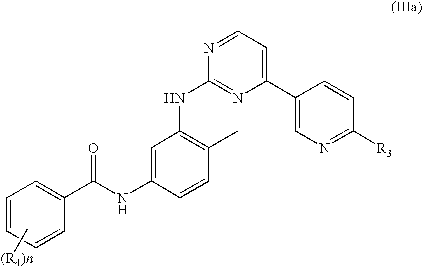

wherein the substituents and preferences are the same as given above for the compounds of formula III.

In another particularly preferred embodiment, the R₃ substitutent is located at the 6-position of the pyridine ring to which it is attached and the R₄ substitutent is attached to the 3-position of the phenyl ring. Thus, the present invention especially includes compounds of the formula (IIIb)

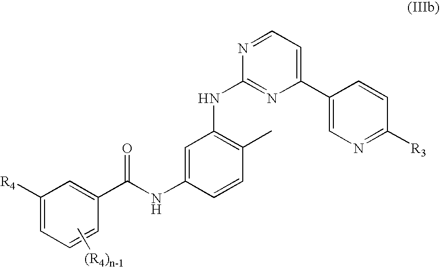

wherein the substituents and preferences are the same as given above for the compounds of formula III.

Another important embodiment of the present invention includes the compounds of formula IV

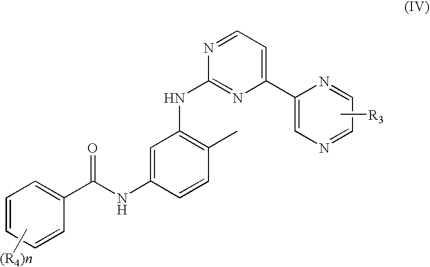

wherein

n is 0, 1 or 2;

R₃ is hydrogen, —NR₅R₆, halogen, —O—R₈, —S—R₈, or lower alkyl which is unsubstituted or substituted by halogen, hydroxy, lower alkoxy, —NR₇R₈, or a heteroaryl or heterocyclic radical attached at a ring carbon;

R₄ is amino, mono- or di-lower alkyl-substituted amino, wherein the alkyl groups are unsubstituted or substituted by halogen or lower alkoxy; halogen, lower alkyl, halo-lower alkyl, lower alkoxy, halo-lower alkoxy, hydroxy, lower alkanoyl, carbamoyl, N-mono- or N,N-di-substituted carbamoyl, mercapto, lower alkylthio or halo-lower alkylthio;

R₅, R₆, R₇ and R₈ are independently hydrogen, a heteroaryl or heterocyclic radical attached at a ring carbon, lower alkyl, C₃-C₈cycloalkyl, C₃-C₈cycloalkyl-lower alkylene, lower alkyl which is substituted by hydroxy, lower alkoxy, a heteroaryl radical, a heterocyclic radical, amino, mono- or di-lower alkyl amino or R₅ and R₆ or R₇ and R₈ together with the nitrogen form a heteroaromatic or heterocyclic radical;

R₈ is a heterocyclic radical, a heteroaromatic radical, heteroaryl-lower-alkylene, heterocyclic-lower-alkylene, lower alkyl or lower alkyl which is substituted by hydroxy, lower alkoxy or —NR₇R₈;

or a pharmaceutically acceptable salt thereof.

In the compounds of formula (IV), at least one R₄ substituent is preferably located at the position on the phenyl ring that is meta to the carbonyl. R₄ is preferably lower alkyl, especially methyl or ethyl; halo-lower alkyl, especially difluoromethyl, trifluoromethyl, 2,2,2-trifluoroethyl, or 1,1,2,2-tetrafluoroethyl; lower alkoxy, especially methoxy or ethoxy; halo-lower alkoxy, especially difluoromethoxy, trifluoromethoxy, 2,2,2-trifluoroethoxy, and 1,1,2,2-tetrafluoroethoxy, lower alkylthio, such as methylmercapto, halo-lower alkylthio, such as difluoromethylthio, trifluoromethylthio, 2,2,2-trifluoroethylthio, or 1,1,2,2-tetrafluoroethylthio.

In an important embodiment of the compounds of formula IV, R₃ is lower alkyl, preferably lower alkyl located at the 3-position of the piperazine ring.

The present invention further relates to compounds of the formula (V).

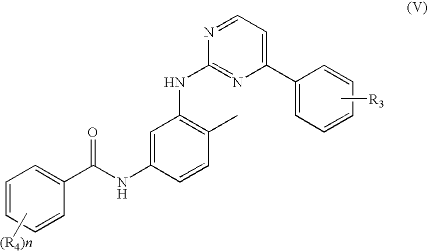

wherein

n is 0, 1 or 2;

R₃ is —NR₅R₆, halogen, —O—R₈, —S—R₈, or lower alkyl which is unsubstituted or substituted by halogen, hydroxy, lower alkoxy, —NR₇R₈, or a heteroaryl or heterocyclic radical attached at a ring carbon;

R₄ is amino, mono- or di-lower alkyl-substituted amino, wherein the alkyl groups are unsubstituted or substituted by halogen or lower alkoxy; halogen, lower alkyl, halo-lower alkyl, lower alkoxy, halo-lower alkoxy, hydroxy, lower alkanoyl, carbamoyl, N-mono- or N,N-di-substituted carbamoyl, mercapto, lower alkylthio or halo-lower alkylthio;

R₅, R₆, R₇ and R₈ are independently hydrogen, a heteroaryl or heterocyclic radical attached at a ring carbon, lower alkyl, C₃-C₈cycloalkyl, C₃-C₈cycloalkyl-lower alkylene, lower alkyl which is substituted by hydroxy, lower alkoxy, a heteroaryl radical, a heterocyclic radical, amino, mono- or di-lower alkyl amino or R₅ and R₆ or R₇ and R₈ together with the nitrogen form a heteroaromatic or heterocyclic radical;

R₈ is a heterocyclic radical, a heteroaromatic radical, heteroaryl-lower-alkylene, heterocyclic-lower-alkylene, lower alkyl or lower alkyl which is substituted by hydroxy, lower alkoxy or —NR₇R₈;

or a pharmaceutically acceptable salt thereof.

In the compounds of formula (V), at least one R₄ substituent is preferably located at the position on the phenyl ring that is meta to the carbonyl. R₄ is preferably lower alkyl, especially methyl or ethyl; halo-lower alkyl, especially difluoromethyl, trifluoromethyl, 2,2,2-trifluoroethyl, or 1,1,2,2-tetrafluoroethyl; lower alkoxy, especially methoxy or ethoxy; halo-lower alkoxy, especially difluoromethoxy, trifluoromethoxy, 2,2,2-trifluoroethoxy, and 1,1,2,2-tetrafluoroethoxy, lower alkylthio, such as methylmercapto, halo-lower alkylthio, such as difluoromethylthio, trifluoromethylthio, 2,2,2-trifluoroethylthio, or 1,1,2,2-tetrafluoroethylthio.

In an important embodiment of the compounds of formula (V), R₃ is lower alkyl or lower alkoxy, especially wherein the substituent is located meta or para to the attachment with the pyrimidine ring.

The patient is a mammal, generally a human, suffering from a disease that is characterized by excessive signaling through the MAP kinase pathway. This can be measured by activation state specific antibodies to pathway members by methods such as Western blot analysis or immunohistochemistry. Such methods are known to those of skill in the art.

In general, the disease characterized by excessive signaling through the MAP kinase signaling pathway is a proliferative disease, particularly a cancer characterized by increased RAF kinase activity, for example one which overexpresses wild-type B- or C-RAF kinase, or that expresses an activating mutant RAF kinase, for example a mutant B-RAF kinase. Cancers wherein a mutated RAF kinase has been detected include melanoma, colorectal cancer, ovarian cancer, gliomas, adenocarcinomas, sarcomas, breast cancer and liver cancer. Mutated B-RAF kinase is especially prevalent in many melanomas.

In accordance with the present invention, a sample of diseased tissue may taken from the patient, for example, as :a result of a biopsy or resection, and tested to determine whether the tissue produces a mutant RAF kinase, such as a mutant B-RAF kinase or overexpresses a wild-type RAF kinase, such as wild-type B- or C-RAF kinase. If the test indicates that mutant RAF kinase is produced or that a RAF kinase Is overproduced in the diseased tissue, the patient is treated by administration of an effective RAF-inhibiting amount of a RAF inhibitor compound described herein.

However, it is also possible to downregulate the MAP kinase signaling pathway with a RAF kinase inhibiting compound if another kinase in the cascade is the cause of excessive signaling in the pathway. Thus, the present invention further relates to the treatment of a disease characterized by excessive signaling in the MAP kinase signaling pathway attributed to a cause other than an activating mutation in or overexpression of a RAF kinase.

Tissue samples are tested by methods generally known in the art. For example, B-RAF mutations are detected by allele specific PCR, DHPLC, mass spectropscopy and overexpression of wild-type B- or C-RAF detected by immunohistochemistry, immunofluorescense, or Western blot analysis. A particularly useful method of detecting B-RAF mutations is the polymerase chain reaction based method described in Example D1. Similar methods are used to determine whether other kinases in the cascade are mutant or overexpressed.

A particularly important aspect of this invention relates to a method of treating melanoma, which comprises (a) testing melanoma tissue from a patient to determine whether the melanoma tissue expresses mutant RAF kinase or overexpresses a wild-type RAF kinase and (b) treating the patent with an effective RAF kinase inhibiting amount of a RAF-inhibiting compound described herein if the melanoma tissue is found to overexpress a wild type RAF kinase or express an activating mutant B-RAF kinase.

An important aspect of this embodiment relates to a method of treating melanoma, which comprises (a) testing melanoma tissue from a patient to determine whether the melanoma tissue overexpresses B-RAF kinase or C-RAF kinase activity and (b) treating the patient with an effective RAF kinase inhibiting amount of a RAF inhibiting compound described herein if the melanoma tissue is found to overexpress the B-RAF kinase or C-RAF kinase activity.

Another important aspect of this embodiment relates to a method of treating melanoma, which comprises (a) testing melanoma tissue from a patient to determine whether the melanoma tissue expresses mutant B-RAF kinase and (b) treating the patient with an effective RAF kinase inhibiting amount of a RAF inhibiting compound described herein if the melanoma tissue is found to express mutant B-RAF kinase.

Generally, the B-RAF kinase mutation is one of those described in the Davies et al article cited. These mutations are summarized in Table 1.

TABLE 1

| B-RAF mutation | protein change |
|---|---|
| G1388A | G463E |
| G1388T | G463V |
| G1394C | G465A |
| G1394A | G465E |
| G1394T | G465V |
| G1403C | G468A |
| G1403A | G468E |
| G1753A | E585K |
| T1782G | F594L |
| G1783C | G595R |
| C1786G | L596V |
| T1787G | L596R |
| T1796A | V599E |
| TG1796-97AT | V599D |

Thus, the present invention particularly relates to a method of treating a disease characterized by an activated mutant B-RAF kinase, which comprises detecting a mutation in the B-RAF kinase gene or protein in a tissue sample from a patient and treating the patient with an effective B-RAF kinase inhibiting compound, especially a compound described herein.

An important aspect of this invention includes those instances wherein the mutant B-RAF kinase exhibits a mutation described in Table 1, especially the V599E mutation.

A particularly important aspect of this invention includes those instances wherein disease is melanoma and the mutant B-RAF kinase exhibits a mutation described in Table 1, especially the V599E mutation.

Accordingly, this invention includes a method of treating a disease characterized by mutant B-RAF kinase, which comprises detecting a mutation in the B-RAF kinase gene selected from G1388A, G1388T, G1394C, G1394A, G1394T, G1403C, G1403A, G1753A, T1782G, G1783C, C1786G, T1787G, T1796A and TG1796-97AT, or corresponding mutation in the RAF kinase protein, in a tissue sample from a patient and treating the patient with an effective B-RAF kinase inhibiting compound described herein.

The present invention further relates to a method of inhibiting RAF kinase, which comprises contacting the RAF kinase with a compound of formula (I), (II), (III), (IV) or (V). Preferably, the RAF kinase is B- or C-RAF kinase, or a mutant RAF kinase, especially a mutant B-RAF kinase, particularly the V599E mutant. The RAF kinase may be isolated or in a cellular environment.

The compounds of formula I (or N-oxides thereof) have valuable pharmacological properties, as described above.

The compounds of the present invention may be administered alone or in combination with other anticancer agents, such as compounds that inhibit tumor angiogenesis, for example, the protease inhibitors, epidermal growth factor receptor kinase inhibitors, vascular endothelial growth factor receptor kinase inhibitors and the like; cytotoxic drugs, such as antimetabolites, like purine and pyrimidine analog antimetabolites; antimitotic agents like microtubule stabilizing drugs and antimitotic alkaloids; platinum coordination complexes; anti-tumor antibiotics; alkylating agents, such as nitrogen mustards and nitrosoureas; endocrine agents, such as adrenocorticosteroids, androgens, anti-androgens, estrogens, anti-estrogens, aromatase inhibitors, gonadotropin-releasing hormone agonists and somatostatin analogues and compounds that target an enzyme or receptor that is overexpressed and/or otherwise involved a specific metabolic pathway that is upregulated in the tumor cell, for example ATP and GTP phosphodiesterase inhibitors, protein kinase inhibitors, such as serine, threonine and tyrosine kinase inhibitors, for example, Abelson protein tryosine kinase and the various growth factors, their receptors and kinase inhibitors therefore, such as, epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors and the like; methionine aminopeptidase inhibitors, proteasome inhibitors, cyclooxygenase inhibitors, for example, cyclooxygenase-1 or -2 inhibitors, and histone deacetylase inhibitors.

The compound of the present invention may also be administered together with radiotherapy, immunotherapy, surgical treatment or combinations thereof. Treatment to maintain the status of a patient after tumor remission or even chemopreventive treatment, for example in the case of at-risk patients, is also possible.

Compounds according to the invention are intended not only for the (prophylactic and, preferably, therapeutic) treatment of human beings, but also for the treatment of other warm-blooded animals, for example of commercially useful animals, for example rodents, such as mice, rabbits or rats, or guinea pigs.

In general, the invention relates also to the use of a compound of formula I (or an N-oxide thereof) in inhibiting RAF kinase activity.

The compounds of the present invention are preferably administered as an active ingredient in a pharmaceutical composition. Preference is given to a pharmaceutical composition which is suitable for administration to a warm-blooded animal, especially a human being or a commercially useful mammal, which is suffering from a disease characterized by an aberrant MAP kinase signaling pathway especially, a tumor disease, most particularly melanoma, comprising a compound of formula I (or an N-oxide thereof), or a pharmaceutically acceptable salt thereof where salt-forming groups are present, in an amount that is effective in inhibiting RAF kinase, particularly a mutant RAF kinase, together with at least one pharmaceutically acceptable carrier.

Preference is given also to a pharmaceutical composition for the prophylactic or, especially, therapeutic treatment of tumor diseases and other proliferative diseases in a warm-blooded animal, especially a human being or a commercially useful mammal, which requires such treatment, especially which is suffering from such a disease, comprising a novel compound of formula I (or an N-oxide thereof), or a pharmaceutically acceptable salt thereof, as active ingredient in an amount that is effective prophylactically or, especially, therapeutically against the mentioned diseases.

Pharmaceutical compositions comprise from approximately 1% to approximately 95% active ingredient, dosage forms that are in single dose form preferably comprising from approximately 20% to approximately 90% active ingredient, and dosage forms that are not in single dose form preferably comprising from approximately 5% to approximately 20% active ingredient. Unit dose forms are, for example, dragees, tablets, ampoules, vials, suppositories or capsules. Other dosage forms are, for example, ointments, creams, pastes, foams, tinctures, lipsticks, drops, sprays, dispersions, etc. Examples are capsules comprising from approximately 0.05 g to approximately 1.0 g of the active ingredient.

The pharmaceutical compositions of the present invention are prepared in a manner known per se, for example by means of conventional mixing, granulating, confectioning, dissolving or lyophilising processes.

Solutions of the active ingredient are preferably used, in addition also suspensions or dispersions, especially isotonic aqueous solutions, dispersions or suspensions, which, in the case of, for example, lyophilised compositions which contain the active substance alone or together with a carrier, for example mannitol, can be prepared prior to use. The pharmaceutical compositions may be sterilised and/or comprise excipients, for example preservatives, stabilisers, wetting agents and/or emulsifiers, solubilisers, salts for regulating the osmotic pressure and/or buffers, and are prepared in a manner known per se, for example by means of conventional dissolving or lyophilising processes. The mentioned solutions or suspensions may comprise viscosity-increasing substances, such as sodium carboxymethylcellulose, carboxymethylcellulose, dextran, polyvinylpyrrolidone or gelatin, or solubilisers, for example Tween 80 [polyoxyethylene(20)sorbitan monooleate; trade mark of ICI Americas, Inc, USA].

Suspensions in oil comprise as the oily component the vegetable, synthetic or semi-synthetic oils customary for -injection purposes. There may be mentioned as such especially liquid fatty acid esters, which comprise as the acid component a long-chained fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brassidic acid or linoleic acid, optionally with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydric, for example mono-, di- or tri-hydric, alcohol, for example methanol, ethanol, propanol, butanol or pentanol or their isomers, but especially glycol and glycerol. Examples of fatty acid esters which may be mentioned are, therefore: ethyl oleate, isopropyl myristate, isopropyl palmitate, “Labrafil M 2375” (polyoxyethyleneglycerol trioleate from Gattefossé, Paris), “Labrafil M 1944 CS” (unsaturated polyglycolised glycerides prepared by alcoholysis of apricot kernel oil and composed of glycerides and polyethylene glycol ester; Gattefossé, France), “Labrasol” (saturated polyglycolised glycerides prepared by alcoholysis of TCM and composed of glycerides and polyethylene glycol ester; Gattefossé, France) and/or “Miglyol 812” (triglyceride of saturated fatty acids having a chain length of from C₈ to C₁₂ from Hüls AG, Germany), but especially vegetable oils, such as cottonseed oil, almond oil, olive oil, castor oil, sesame oil, soybean oil and, more especially, groundnut oil.

The preparation of the injection compositions is carried out in customary manner under sterile conditions, as are also the introduction thereof, for example, into ampoules or vials and the sealing of the containers.

Pharmaceutical compositions for oral administration can be obtained, for example, by combining the active ingredient with one or more solid carriers, granulating a resulting mixture, where appropriate, and processing the mixture or granules, if desired, where appropriate by addition of additional excipients, to tablets or dragée cores.

Suitable carriers are especially fillers, such as sugars, for example lactose, saccharose, mannitol or sorbitol, cellulose preparations and/or calcium phosphates, for example tri-calcium phosphate or calcium hydrogen phosphate, also binders, such as starches, for example corn, wheat, rice or potato starch, methylcellulose, hydroxypropylmethylcellulose, sodium carboxymethylcellulose and/or polyvinylpyrrolidone, and/or, if desired, disintegrators, such as the above-mentioned starches, also carboxymethyl starch, crosslinked poly-vinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Dragée cores can be provided with suitable, optionally enteric, coatings, there being used inter alia concentrated sugar solutions which may contain gum arabic, talc, polyvinylpyrrolidone, polyethylene glycol and/or titanium dioxide, or coating solutions in suitable organic solvents or solvent mixtures or, for the preparation of enteric coatings, solutions of suitable cellulose preparations, such as acetylcellulose phthalate or hydroxypropylmethylcellulose phthalate. Colourings or pigments may be added to the tablets or dragée coatings, for example for identification purposes or to indicate different doses of active ingredient.

Pharmaceutical compositions for oral administration are also hard gelatin capsules and soft sealed capsules consisting of gelatin and a plasticiser, such as glycerol or sorbitol. The hard gelatin capsules may contain the active ingredient in the form of granules, for example in admixture with fillers, such as corn starch, binders and/or glidants, such as talc or magnesium stearate, and optionally stabilisers. In soft capsules the active ingredient is preferably dissolved or suspended in suitable liquid excipients, such as fatty oils, paraffin oil or liquid polyethylene glycols or fatty acid esters of ethylene glycol or propylene glycol, it likewise being possible to add stabilisers and detergents, for example of the polyoxyethylenesorbitan fatty acid ester type.

Suitable rectally administrable pharmaceutical compositions are, for example, suppositories that consist of a combination of the active ingredient with a suppository base. Suitable suppository bases are, for example, natural or synthetic triglycerides, paraffin hydrocarbons, polyethylene glycols or higher alkanols.

For parenteral administration there are suitable, especially, aqueous solutions of an active ingredient in water-soluble form, for example in the form of a water-soluble salt, or aqueous injection suspensions that comprise viscosity-increasing substances, for example sodium carboxymethylcellulose, sorbitol and/or dextran and, if desired, stabilisers. The active ingredient, optionally together with excipients, can also be in the form of a lyophilisate and can be made into a solution prior to parenteral administration by the addition of suitable solvents.

Solutions used, for example, for parenteral administration can also be used as infusion solutions.

Preferred preservatives are, for example, antioxidants, such as ascorbic acid, or microbicides, such sorbic acid or benzoic acid.

The invention relates especially to a process or a method for treating one of the pathological conditions that is characterized by an aberrant MAP kinase signaling pathway, especially a disease responsive to inhibition of RAF kinase, especially a corresponding tumor disease. The compounds of formula I (or an N-oxide thereof) can be administered prophylactically or therapeutically as such or in the form of pharmaceutical compositions, preferably in an amount that is effective against the mentioned diseases, to a warm-blooded animal, for example a human being, requiring such treatment, the compounds being used especially in the form of pharmaceutical compositions. In the case of a body weight of approximately 70 kg, a daily dose of from approximately 0.1 g to approximately 5 g, preferably from approximately 0.5 g to approximately 2 g, of a compound of the present invention is administered.

The preferred dosage, composition and preparation of pharmaceutical formulations (medicaments) to be used in each particular case are described above.

The compounds of the present invention are prepared utilizing methods known to those of ordinary skill in the art according to the general reaction schemes described below.
Step 1

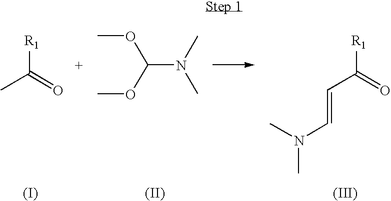

A mixture of I and II are heated to 85° C. After stirring overnight, the mixture is cooled first in air then in an ice/water bath and filtered. The resulting solid is washed well with ether and dried to yield III
Preparation of Common Intermediate VII

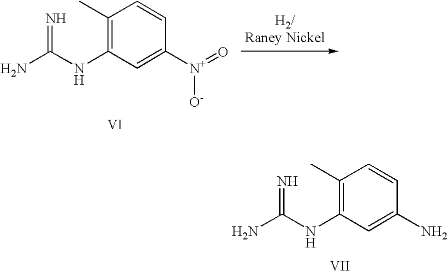

Raney nickel is charged to a degassed solution of VI in methanol (100 mL) and covered with hydrogen (60 psi.). The mixture is shaken overnight and filtered. Concentration in vacuo yields VII as a brown solid.
Reaction of III and VII

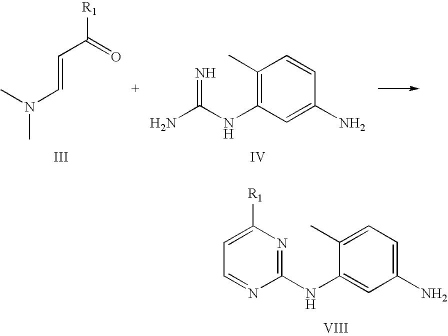

III and IV are dissolved in 2-propanol with heating at about 85° C. After stirring for 24 hours, the mixture is cooled and filtered. The resulting solid is washed well with ether and dried to yield V.

Final Step

V is reacted with an appropriately substituted benzoic acid or benzoic acid halide under standard conditions to yield the final benzamide.

The present invention further relates to the compounds of formula VII and VIII as intermediates for the preparation of the compounds of formulae (I), II, II, III, III(a), III(b) IV and V.

Alternative Synthetic Scheme

The alternative synthetic scheme discloses the synthesis of a common intermediate for the preparation of compounds of formula III wherein R₄ is 3-trifluoromethyl. One of skill in the art has the ability to substitute various starting materials in order to prepare the inventive compounds.

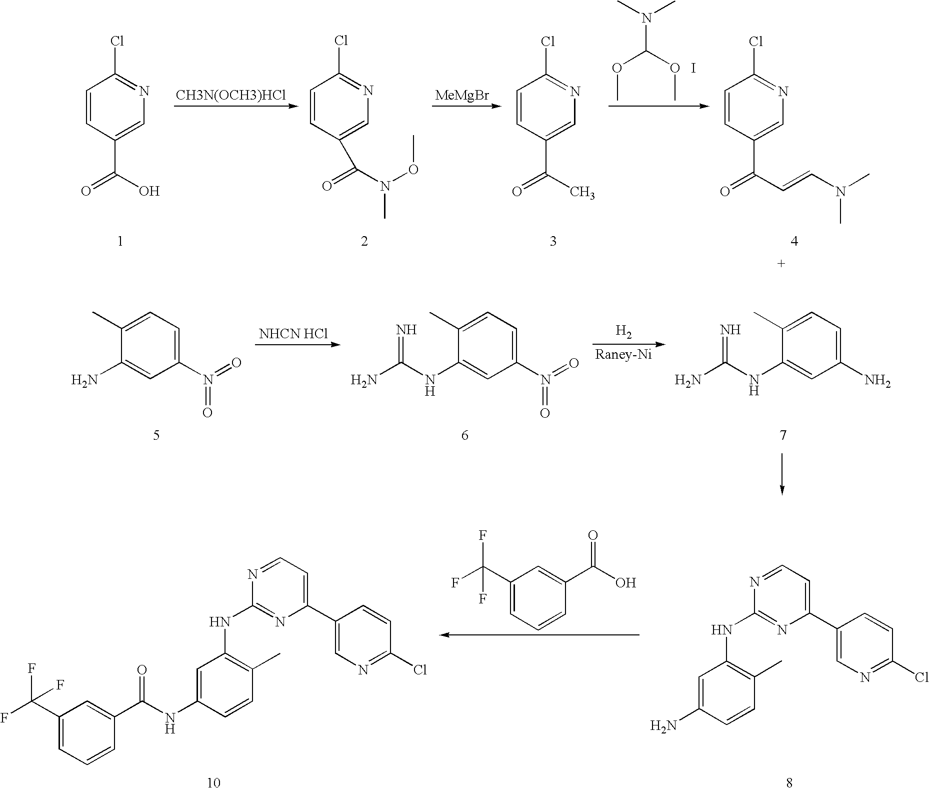

The present invention further relates to the compound of formula 8 as an intermediate for the preparation of the compounds of formula III, III(a) and III(b).

The Examples which follow serve to illustrate the invention without limiting the scope thereof.

### EXAMPLE 1

### N-[4-Methyl-3-(4-thiazol-2-yl-pyrimidin-2-ylamino)-phenyl]-3-trifluoromethyl-benzamide

A mixture of 4-Methyl-N*3*-(4-thiazol-2-yl-pyrimidin-2-yl)-benzene-1,3-diamine (0.17 g, 0.60 mmol), 3-Trifluoromethyl-benzoic acid (0.12 g, 0.63 mmol), BOP (0.32 g, 0.72 mmol) and DIEA (0.4 mL, 2.16 mmol) is stirred in 2.5 mL of DMF at room temperature, under nitrogen overnight. After 17.5 hours of stirring, 20 mL of deionized water is added to the precipitate. The product and the mixture are allowed to stir at room temperature for 45 minutes. They are then filtered through a Buchner funnel, triturated with 10.0 mL of CH₃OH, stirred for 15 minutes, filtered again, rinsed with 10.0 mL of CH₃OH and dried. 0.11 g of N-[4-Methyl-3-(4-thiazol-2-yl-pyrimidin-2-ylamino)-phenyl]-3-trifluoromethyl-benzamide is obtained as a yellow solid.

¹H NMR (DMSO, 500 MHz): δ (ppm) 10.41(s, 1H), 9.17 (s, 1H), 8.64 (d, 1H, J=5.05 Hz), 8.29 (br m, 1H), 8.26 (d, 1H, J=7.88 Hz), 8.06 (d, 1H, J=3.15 Hz), 7.96 (br m, 1H), 7.94 (d, 1H, J=3.16 Hz), 7.91 (1H, d, J=2.27 Hz), 7.77 (t, 1H, J=7.88 Hz), 7.56 (dd, 1H, J₁=8.35 Hz, J₂=2.45 Hz), 7.39 (d, 1H, J=4.39 Hz), 7.24 (d, 1H, J=8.79 Hz), 2.22 (s, 3H).

API-MS m/z 456.1 ([M+H]⁺, calcd 456.1)

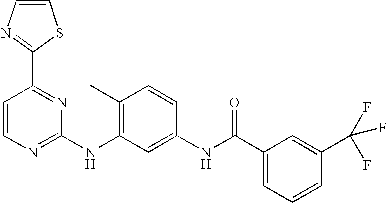

### EXAMPLE 2

### N-[4-Methyl-3-(4-pyrazin-2-yl-pyrimidin-2-ylamino)-phenyl]-3-trifluoromethyl-benzamide

A mixture of 4-Methyl-N*3*-(4-pyrazin-2-yl-pyrimidin-2-yl)-benzene-1,3-diamine (0.36 g, 1.30 mmol), 3-Trifluoromethyl-benzoic acid (0.26 g, 1.36 mmol), BOP (0.69 g, 1.56 mmol) and DIEA (0.8 mL, 4.68 mmol) is stirred in 5.0 mL of DMF at room temperature, under Nitrogen overnight. 50 mL of deionized water is added to the resulting precipitate. The mixture is allowed to stir at room temperature for 45 minutes and then is filtered through a Buchner funnel, triturated with 25.0 mL of CH₃OH, stirred for 15 minutes, filtered again, rinsed with 25.0 mL of CH₃OH and dried. 0.41 g of N-[4-Methyl-3-(4-pyrazin-2-yl-pyrimidin-2-ylamino)-phenyl]-3-trifluoromethyl-benzamide is obtained as a pale yellow solid.

¹H NMR (DMSO, 500 MHz): δ (ppm) 10.45 (s, 1H), 9.49 (s, 1H), 9.10 (s,1H), 8.79 (s, 2H), 8.63 (d, 1H, J=5.39 Hz), 8.31 (br m, 1H), 8.28 (d, 1H, J=8.08 Hz), 8.16 (d, 1H, J=2.37 Hz), 7.96 (d, 1H, J=8.08 Hz), 7.79 (t, 1H, J=8.08 Hz), 7.63 (d, 1H, J=4.87 Hz), 7.50 (dd, 1H, J₁=8.67 Hz, J₂=2.17 Hz), 7.26 (d, 1H, J=7.59 Hz), 2.26 (s, 3H).

API-MS m/z 451.1 ([M+H]⁺, calcd 451.1)

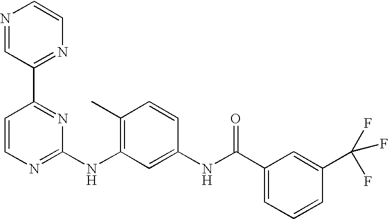

### EXAMPLE 3

### N-[4-Methyl-3-(4-pyrazin-2-yl-pyrimidin-2-ylamino)-phenyl]-3-(1,1,2,2-tetrafluoro-ethoxy)-benzamide

A mixture of 4-Methyl-N*3*-(4-pyrazin-2-yl-pyrimidin-2-yl)-benzene-1,3-diamine (0.55 g, 1.98 mmol), 3-(1,1,2,2-Tetrafluoro-ethoxy)-benzoic acid (0.50 g, 2.08 mmol), BOP (1.05 g, 2.38 mmol) and DIEA (1.25 mL, 7.14 mmol) is stirred in 10.0 mL of DMF at room temperature for 20 hours. 50 mL of deionized water is added to the resulting precipitate. The resulting suspension is allowed to stir at room temperature for 45 minutes. The mixture is then filtered through a Buchner funnel, triturated with 20.0 mL of CH₃OH, stirred for 15 minutes., filtered again, rinsed with 20.0 mL of CH₃OH and dried. 0.71 g of N-[4-Methyl-3-(4-pyrazin-2-yl-pyrimidin-2-ylamino)-phenyl]-3-(1,1,2,2-tetrafluoro-ethoxy)-benzamide is obtained as a pale. yellow solid.

¹H NMR (DMSO, 500 MHz): δ (ppm) 10.36 (s, 1H), 9.49 (s, 1H), 9.10 (s, 1H), 8.79 (s,2H), 8.62 (d, 2H, J=5.13 Hz), 8.15 (br m, 1H), 7.99 (d, 2H, J=8.33 Hz), 7.86 (br m, 1H), 7.65(m, 2H), 7.51 (m, 2H), 7.25 (d, 1H,J=8.4 Hz), 6.86 (t, 1H, J=52.4 Hz), 2.26 (s, 3H).

API-MS m/z 499.4 ([M+H]⁺, calcd 499.4)

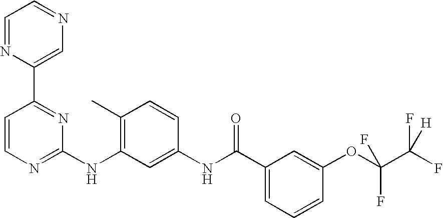

### EXAMPLE 4

### 3-Difluoromethoxy-N-[4-methyl-3-(4-pyrazin-2-yl-pyrimidin-2-ylamino)-phenyl]-benzamide

A mixture of 4-Methyl-N*3*-(4-pyrazin-2-yl-pyrimidin-2-y) -benzene-1,3-diamine (0.55 g, 1.98 mmol), 3-(2,2-Difluoro-ethoxy)-benzoic acid (0.39 g, 2.08 mmol), BOP (1.05 g, 2.38 mmol) and DIEA (1.25 mL, 7.14 mmol) is stirred in 10.0 mL of DMF at room temperature overnight. 50 mL of deionized water is added to the precipitate. The resulting suspension is allowed to stir at room temperature for 45 minutes. It is then filtered through a Buchner funnel, triturated with 20.0 mL of CH₃OH, stirred for 15 minutes, filtered again, rinsed with 20.0 mL of CH₃OH and dried. 0.79 g of 3-3-Difluoromethoxy-N-[4-methyl-3-(4-pyrazin-2-yl-pyrimidin-2-ylamino)-phenyl]-benzamide is obtained as a pale yellow solid.

¹H NMR (DMSO, 500 MHz): δ (ppm) 10.29 (s, 1H), 9.47 (s, 1H), 9.09 (s, 1H), 8.79 (s,1H), 8.61 (d, 1H, J₁=5.45 Hz, J₂=1.82 Hz), 8.14 (m, 1H), 7.85 (d, 1H, J=7.61 Hz), 7.64-7.56 (m, 2H), 7.51-7.46 (m, 1H), 7.40(dd, 1H, J₁=7.62 Hz, J₂=2.77 Hz), 7.33 (t, 1H, J=74.2 Hz), 7.24 (d, 1H, J=8.32 Hz), 2.25(s, 3H).

API-MS m/z 449.3 ([M+H]⁺, calcd 449.4)

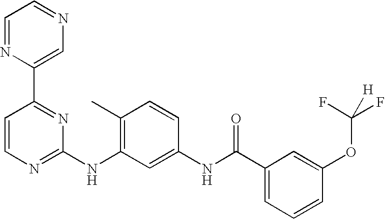

### EXAMPLE 5

### 3-Dimethylamino-N-[4-methyl-3-(4-pyrazin-2-yl-pyrimidin-2-ylamino)-phenyl]-benzamide

A mixture of 4-Methyl-N*3*-(4-pyrazin-2-yl-pyrimidin-2-yl)-benzene-1,3-diamine (0.55 g, 1.98 mmol), 3-Dimethylamino-benzoic acid (0.34 g, 2.08 mmol), BOP (1.05 g, 2.38 mmol) and DIEA (1.25 mL, 7.14 mmol) is stirred in 10.0 mL of DMF at room temperature for 20 hours. 50 mL of deionized water is added to the precipitate. The resulting suspension is allowed to stir at room temperature for 45 minutes. It is then filtered through a Buchner funnel, triturated with 20.0 mL of CH₃OH, stirred for 15 minutes, filtered again, rinsed with 20.0 mL of CH₃OH and dried. 0.64 g of 3-Dimethylamino-N-[4-methyl-3-(4-pyrazin-2-yl-pyrimidin-2-ylamino)-phenyl]-benzamide is obtained as an off-white solid.

¹H NMR (DMSO, 500 MHz): δ (ppm) 10.09 (s, 1H), 9.47 (s, 1,H), 9.07 (s, 1H), 8.79 (s,1H), 8.61 (d, 1H, J=5.45 Hz,), 8.14 (m, 1H), 7.62 (d, 1H, J=5.52 Hz), 7.50 (dd, 1H, J₁=8.47 Hz, J₂=2.31 Hz), 7.31 (dd, 1H, J=8.08 Hz, J=8.08 Hz), 7.24 (m,2H), 7.21 (m, 1H),6.92 (dd, 1H, J₁=8.48 Hz, J₂=2.31 Hz), 2.97 (s, 6H), 2.25 (s, 3H).

API-MS m/z 426.1 ([M+H]⁺, calcd 426.2)

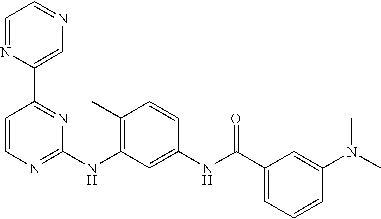

### EXAMPLE 6

### N-[4-Methyl-3-(4-pyrazin-2-yl-pyrimidin-2-ylamino)-phenyl]-3-(2,2,2-trifluoro-ethoxy)-benzamide

A mixture of 4-Methyl-N*3*-(4-pyrazin-2-yl-pyrimidin-2-yl)-benzene-1,3-diamine (0.55 g,1.98 mmol), 3-(2,2,2-Trifluoro-ethoxy)-benzoic acid (0.46 g, 2.08 mmol), BOP (1.05 g, 2.38 mmol) and DIEA (1.25 mL, 7.14 mmol) is stirred in 10.0 mL of DMF at room temperature for 20 hours. 50 mL of deionized water is added to the precipitate. The resulting suspension is allowed to stir at room temperature for 45 minutes. It is then filtered through a Buchner funnel, triturated with 25.0 mL of CH₃OH, stirred for 15 minutes, filtered again, rinsed with 25.0 mL of CH₃OH and dried. 0.63 g of N-[4-Methyl-3-(4-pyrazin-2-yl-pyrimidin-2-ylamino)-phenyl]-3-(2,2,2-trifluoro-ethoxy)-benzamide is obtained as a light yellow solid.

¹H NMR (DMSO, 300 MHz): δ (ppm) 10.21 (s, 1H), 9.48 (s, 1H), 9.10 (s, 1H), 8.79 (s,2H), 8.62 (d, 1H, J=4.82 Hz), 8.15 (br m, 1H), 7.63 (m, 3H), 7.50 (m, 2H), 7.29 (dd, 1H, J₁=8.05 Hz, J₂=2.15 Hz), 7.24 (d, 1H, J=8.58 Hz), 4.86(q, 2H,J=8.8 Hz), 2.25 (s, 3H).

API-MS m/z 481.3 ([M+H]⁺, calcd 481.1)

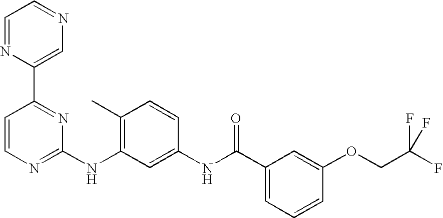

### EXAMPLE 7

### N-[4-Methyl-3-(4-pyrazin-2-yl-pyrimidin-2-ylamino)-phenyl]-3-trifluoromethylsulfanyl-benzamide

A mixture of 4-Methyl-N*3*-(4-pyrazin-2-yl-pyrimidin-2-yl)-benzene-1,3-diamine (0.55 g, 1.98 mmol), 3-Trifluoromethylsulfanyl-benzoic acid (0.46 g, 2.08 mmol), BOP (1.05 g, 2.38 mmol) and DIEA (1.25 mL, 7.20 mmol) is stirred in 6.0 mL of DMF at room temperature overnight. 50 mL of water is added to the precipitate. The product and the mixture are allowed to stir at room temperature for one hour and then are filtered through a Buchner funnel, air dried and triturated with 10.0 mL of CH₃OH for 45 minutes and dried. 0.78 g of -N-[4-Methyl-3-(4-pyrazin-2-yl-pyrimidin-2-ylamino)-phenyl]-3-trifluoromethylsulfanyl-benzamide is obtained as a yellow solid.

¹H NMR (DMSO, 500 MHz): δ (ppm) 10.40 (s,1H), 9.48 (s, 1H), 9.10 (s, 1H), 8.79(s, 2H), 8.62 (d, 1H, J=5.38 Hz), 8.30 (br m, 1H), 8.20(d, 1H, J=7.68 Hz), 8.15 (m,1H), 7.94(d, 1H, J=7.68 Hz), 7.72 (t, 1H, J=8.06 Hz), 7.63 (d, 1H, J=5.38 Hz), 7.50 (dd, 1H, J₁=8.44 Hz, J₂=2.29 Hz), 7.25 (d, 1H, J=8.44 Hz), 2.26 (s, 3H).

API-MS m/z 483.1 ([M+H]⁺, calcd 483.1)

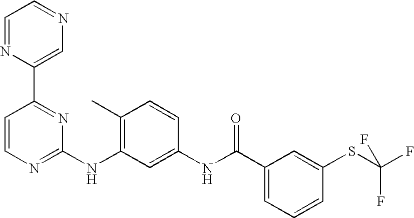

### EXAMPLE 8

### N-{3-[4-(4-Methoxy-phenyl)-pyrimidin-2-ylamino]-4-methyl-phenyl}-3-trifluoromethyl-benzamide

A mixture of N*3*-[4-(4-Methoxy-phenyl)-pyrimidin-2-yl]-4-methyl-benzene-1,3-diamine (0.29 g, 0.95 mmol), 3-Trifluoromethyl-benzoic acid (0.19 g, 0.99 mmol), BOP (0.50 g, 1.14 mmol) and DIEA (0.6 mL, 3.42 mmol) is stirred in 5.0 mL of DMF at room temperature overnight. 50 mL of water is added to the precipitate. The product and the mixture are allowed to stir at room temperature for one hour then are filtered through a Buchner funnel, air dried and triturated with 6.0 mL of CH₃OH for 45 minutes and dried. 0.38 g of N-3-[4-(4-Methoxy-phenyl)-pyrimidin-2-ylamino]-4-methyl-phenyl)-3-trifluoromethyl-benzamide is obtained as a yellow solid.

¹H NMR (DMSO, 500 MHz): δ (ppm) 10.43 (s, 1H), 8.40 (d, 2H, J=5.21 Hz), 8.31 (s, 1H), 8.28(d, 1H, J=7.89 Hz), 8.13 (s, 1H), 8.11 (m, 2H, br),7.96(d, 1H, J=8.35 Hz), 7.79(t, 1H,J=7.72 Hz), 7.50 (d, 1H, J=8.20 Hz), 7.28(d, 1H, J=5.21 Hz), 7.23(d, 1H, J=8.35 Hz), 7.02 (d, 1H, J=8.99 Hz), 3.81 (s, 3H),2.26 (s, 3H).

API-MS m/z 479.2 ([M+H]⁺, calcd 479.2)

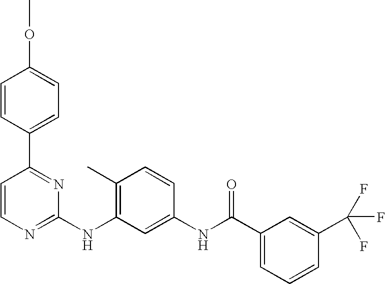

### EXAMPLE 9

### N-[4-Methyl-3-(4-phenyl-pyrimidin-2-ylamino)-phenyl]-3-trifluoromethyl-benzamide

A mixture of 4-Methyl-N*3*-(4-phenyl-pyrimidin-2-yl)-benzene-1,3-diamine (0.50 g, 1.81 mmol), 3-Trifluoromethyl-benzoic acid (0.36 g, 1.90 mmol), BOP (0.96 g, 2.17 mmol) and DIEA (1.25 mL, 6.51 mmol) is stirred in 6.0 mL of DMF at room temperature overnight. 50 mL of water is added to the precipitate. The product and the mixture are allowed to stir at room temperature for one hour, then are filtered through a Buchner funnel, air dried and triturated with 10.0 mL of CH₃OH for 45 minutes and dried. 0.27 g of a yellow solid is obtained. This product is again triturated with 5.0 mL of CH₃OH for 1 hour, filtered and dried. 0.12 g of N-[4-Methyl-3-(4-phenyl-pyrimidin-2-ylamino)-phenyl]-3-trifluoromethyl-benzamide is obtained as a pale yellow solid.

¹H NMR (DMSO, 500 MHz): δ (ppm) 10.43 (s, 1H), 8.90 (s, 1H), 8.48 (d, 1H, J=4.94 Hz), 8.30(m,1H), 8.27 (d, 1H, J=7.40 Hz), 8.13-8.16 (m, 2H), 8.09 (d, 1H, J=2.19), 7.97 (d, 1H, J=7.11 Hz), 7.79(t, 1H,J=7.66 Hz), 7.48-7.52(m, 2H), 7.36 (d, 1H, J=5.47 Hz), 7.24 (d, 1H, J=8.20 Hz), 2.26 (s, 3H).

API-MS m/z 449.3 ([M+H]⁺, calcd 449.4)

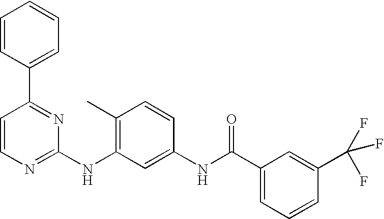

### EXAMPLE 10

### N-{3-[4-(3-Methoxy-phenyl)-pyrimidin-2-ylamino]-4-methyl-phenyl}-3-trifluoromethyl-benzamide

A mixture of N*3*-[4-(3-Methoxy-phenyl)-pyrimidin-2-yl-4-methyl-benzene-1,3-diamine (0.40 g, 1.30 mmol), 3-Trifluoromethyl-benzoic acid (0.26 g, 1.36 mmol), BOP (0.69 g, 1.56 mmol) and DIEA (0.8 mL, 4.68 mmol) is stirred in 5.0 mL of DMF at room temperature overnight. 50 mL of water is added to the precipitate. The mixture is allowed to stir at room temperature for one hour. They are then filtered through a Buchner funnel, air dried and triturated with 10.0 mL of CH₃OH for 45 minutes and dried. 0.34 g of N-{3-[4-(3-Methoxy-phenyl)-pyrimidin-2-ylamino]-4-methyl-phenyl}-3-trifluoromethyl-benzamide is obtained as an off-white solid.

¹H NMR (DMSO, 500 MHz): δ (ppm) 10.43 (s, 1H), 8.90 (s, 1H), 8.48 (d, 1H,J=4.94 Hz), 8.30(m,1H), 8.27 (d, 1H, J=8.40 Hz), 8.11 (m, 1H), 7.97 (d, 1H, J=7.39 Hz), 7.79(t, 1H,J=8.02 Hz), 7.71-7.67 (br m, 2H), 7.51(dd, 1H,J₁=8.41 Hz, J₂=2.00 Hz), 7.40 (t,1H, J=8.20 Hz), 7.36(d, 1H, J=5.21 Hz), 7.07(d,1H,J₁=8.40 Hz, J₂=2.40 Hz), 3.74(s,3H), 2.26 (s, 3H).

API-MS m/z 479.2 ([M+H]⁺, calcd 479.2)

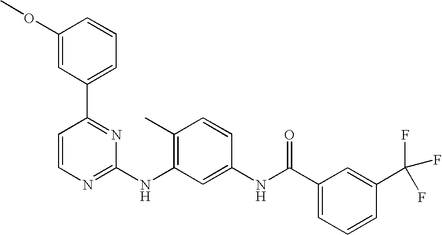

### EXAMPLE 11

### 3-Diethylamino-N-[4-methyl-3-(4-pyrazin-2-yl-pyrimidin-2-ylamino)-phenyl]-benzamide

A mixture of 4-Methyl-N*3*-(4-pyrazin-2-yl-pyrimidin-2-yl)-benzene-1,3-diamine (0.55 g, 1.98 mmol), 3-Diethylamino-benzoic acid (0.48 g, 2.08 mmol), HCl, BOP (1.05 g, 2.38 mmol) and DIEA (1.7 mL, 9.50 mmol) is stirred in 6.0 mL of DMF at room temperature overnight. 50 mL of water is added to the precipitate. The resulting suspension is allowed to stir at room temperature for one hour. The reaction mixture is then filtered through a Buchner funnel, air dried and triturated with 10.0 mL of CH₃OH for 1 hour and dried. 0.87 g of 3-Diethylamino-N-[4-methyl-3-(4-pyrazin-2-yl-pyrimidin-2-ylamino)-phenyl]-benzamide is obtained as a pale yellow solid.

¹H NMR (DMSO, 500 MHz): δ (ppm) 10.07 (s, 1H), 9.50 (s, 1H), 9.07 (s, 1H), 8.78 (s, 2H), 8.62(d, 1H, J=4.78 Hz), 8.17 (m, 1H), 7.62 (d, 1H, J=5.76 Hz), 7.50(dd, 1H, J₁=8.19 Hz, J₂=2.16 Hz), 7.29-7.21 (br m, 2H), 7.17-7.11 (br m, 2H), 6.84 (dd, 1H, J₁=7.8 Hz, J₂=2.44 Hz), 3.39(q, 4H, J=5.99 Hz), 2.25(s,3H), 1.11 (t, 6H, J=6.99 Hz).

API-MS m/z 454.1 ([M+H]⁺, calcd 454.2)

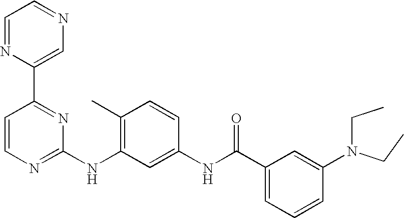

### EXAMPLE 12

### N-[4-Methyl-3-(4-pyrazin-2-yl-pyrimidin-2-ylamino)-phenyl]-3-trifluoromethoxy-benzamide

A mixture of 4-Methyl-N*3*-(4-pyrazin-2-yl-pyrimidin-2-yl)-benzene-1,3-diamine (0.55 g, 1.98 mmol), 3-Trifluoromethoxy-benzoic acid (0.43 g, 2.08 mmol), BOP (1.05 g, 2.38 mmol) and DIEA (1.25 mL, 7.20 mmol) is stirred in 6.0 mL of DMF at room temperature overnight. 50 mL of water is added to the precipitate. The mixture is allowed to stir at room temperature for one hour. The mixture is then filtered through a Buchner funnel, air dried and triturated with 10.0 mL of CH₃OH for 45 minutes and dried. 0.91 g of N-[4-Methyl-3-(4-pyrazin-2-yl-pyrimidin-2-ylamino)-phenyl]-3-trifluoromethoxy-benzamide is obtained as a pale yellow solid.

¹H NMR (DMSO, 500 MHz): δ (ppm) 10.37 9s, 1H), 9.49 9s, 1H), 9.10 (s, 1H), 8.79 (s, 2H), 8.62 (d, 1H, J=5.24 Hz), 8.16 (m, 1H), 8.03 (d, 1H, J=8.09), 7.93 (br s, 1H),7.69 (t, 1H, J=8.02 Hz), 7.63 (d, 1H, 5.15 Hz), 7.60 (br d, 1H, J=8.48 Hz),7.50 (dd, 1H, J₁=8.17 Hz, J₂=2.12 Hz), 7.25 (d, 1H, J=8.48 Hz), 2.26 (s, 3H).

API-MS m/z 467.2 ([M+H]⁺, calcd 467.1)

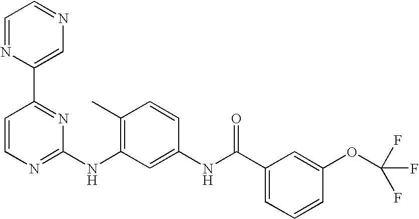

### EXAMPLE 13

### N-{3-[4-(3-Ethyl-pyrazin-2-yl)-pyrimidin-2-ylamino]-4-methyl-phenyl}-3-trifluoromethyl-benzamide

A mixture of N*3*-[4-(3-Ethyl-pyrazin-2-yl)-pyrimidin-2-yl]-4-methyl-benzene-1,3-diamine (0.25 g, 0.82 mmol), 3-Trifluoromethyl-benzoic acid (0.16 g, 0.86 mmol), BOP (0.43 g, 0.98 mmol) and DIEA (0.5 mL, 2.95 mmol) is stirred in 3.0 mL of DMF at room temperature overnight. 50 mL of water is added to the precipitate. The resulting suspension is allowed to stir at room temperature for one hour. It is then filtered through a Buchner funnel, air dried and triturated with 2.0 mL of CH₃OH for 1 hour and dried. 0.18 g of N-{3-[4-(3-Ethyl-pyrazin-2-yl)-pyrimidin-2-ylamino]-4-methyl-phenyl}-3-trifluoromethyl-benzamide is obtained as an off-white solid.

¹H NMR (DMSO, 500 MHz): δ (ppm) 10.40 (s, 1H), 9.05 (s, 1H), 8.64 (d, 1H, J=2.34 Hz), 8.56 (m,2H), 8.28 (m, 1H), 8.25 (d, 1H, J=8.47 Hz), 7.96 (d, 1H, J=8.09 Hz), 7.87(d, 1H,J=2.30 Hz), 7.78 (t, 1H, J=7.36 Hz), 7.53 (dd, 1H,J₁=8.82 Hz,J₂=2.30 Hz), 7.23 (m, 2H), 2.97(q,2H,J=5.8 Hz), 2.22(s,3H), 1.01(t, 3H, J=8.7 Hz).

API-MS m/z 479.5 ([M+H]⁺, calcd 479.5)

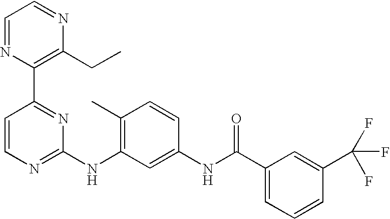

### EXAMPLE 14

### N-(4-Methyl-3-{4-[6-(3-morpholin-4-yl-propylamino)-pyridin-3-yl]-pyrimidin-2-ylamino}-phenyl)-3-trifluoromethyl-benzamide

N-{3-[4-(6-Chloro-pyridin-3-yl)-pyrimidin-2-ylamino]-4-methyl-phenyl}-3-trifluoromethyl-benzamide (500 mg, 1.03 mmoles) is stirred in 5 mL of 3-Morpholin-4-yl-propylamine at 110° C. overnight. The reaction mixture is diluted with water. The yellow precipitate is collected by filtration, triturated with dichloromethane and diethyl ether and dried under vacuum. A white solid is obtained (275mg, yield 45.2%).

¹H NMR (DMSO, 300 MHz): δ (ppm) 10.43 (s, 1H), 9.40 (s, 1H), 8.76 (d, J=2.26 Hz, 1H), 8.69 (s,1H), 8.32 (d, J=5.27 Hz, 1H), 8.27 (d, J=9.80, 2H), 8.10 (d, J=2.26, 1H), 8.07 (m, 1H), 7.96 (d, J=7.9 Hz, 1H), 7.78 (t, J=7.72 Hz, 1H), 7.47(dd, J=2.02, 8.29 Hz, 1H), 7.21 (d, J=13.19 Hz, 1H), 7.20 (s, 1H), 7.12 (t, J=5.27 Hz, 1H), 6.50 (d, J=9.04 Hz, 1H), 3.57-3.54 (m, 4H), 3.35-3.25 (m, 2H), 2.40-2.25 (m, 6H), 2.23 (s, 3H), 1.68 (m, 2H)

API-MS m/z 592.3([M+H]⁺, calcd 592.3)

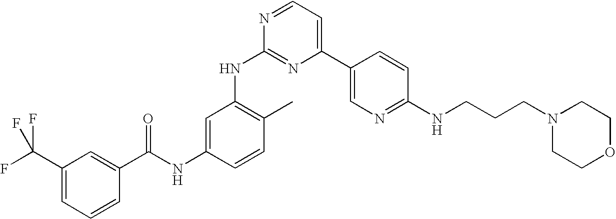

### EXAMPLE 15

### N-(4-Methyl-3-{4-[6-(pyridin4-ylamino)-pyridin-3-yl]-pyrimidin-2-ylamino}-phenyl-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) and Pyridin-4-ylamine (0.5 g, 5.3 mmoles) are dissolved in 1 mL of DMSO at room temperature, then heated to 110° C. and stirred overnight. The reaction mixture is diluted with water. The precipitate is collected by filtration, triturated with dichloromethane and methanol and dried under vacuum. A white solid is obtained (8.5 mg, yield 15.2%).

¹H NMR (DMSO, 300 MHz): δ (ppm) 10.49 (s, 1H), 9.33 (d, J=2.26 Hz, 1H), 9.10 (s, 1H), 8.91 (d, J=7.54 Hz,2H), 8.875-8.80 (m, 3H), 8.59 (d, J=4.90, 1H), 8.29 (d, J=7.9 Hz, 2H), 8.15 (d, J=1.88 Hz, 1H), 8.08(d, J=8.67 Hz, 1H), 7.97 (t, J=7.53 Hz, 1H), 7.79 (t, J=7.54 Hz, 1H), 7.56 (d, J=5.27 Hz, 1H), 7.49 (dd, J=8.29, 1.88 Hz, 1H), 7.25 (d, J=8.29 Hz, 1H), 7.02 (d, J=7.91 Hz, 2H), 2.23 (s, 3H).

API-MS m/z 542.4([M+H]⁺, calcd 542.5)

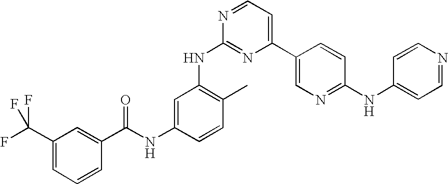

### EXAMPLE 16

### 3-Difluoromethoxy-N-(3-{4-[6-(2-hydroxy-ethylamino)-pyridin-3-yl]-pyrimidin-2-ylamino}-4-methyl-phenyl)-benzamide

N-{3-[4-(6-Chloro-pyridin-3-yl)-pyrimidin-2-ylamino]-4-methyl-phenyl}-3-difluoromethoxy-benzamide (160 mg, 0.331 mmoles) is stirred in 2 ml of 2-Amino-ethanol at 110° C. overnight. The reaction mixture is diluted with water and extracted with ethyl acetate. The combined organic phase is concentrated. The dark residue triturated with a mixture of dichloromethane and diethyl ether and methanol and dried under vacuum. A light yellow solid is obtained (66mg, yield 39.3%).

¹H NMR (DMSO, 300 MHz): δ (ppm) 10.31 (s, 1H), 8.75 (d, J=2.26 Hz, 1H), 8.69 (s,1H), 8.32 (d, J=5.65 Hz, 1H), 8.09 (dd, J=9.61, 2.64 Hz, 2H), 7.86 (d, J=7.91 Hz, 1H), 7.65-7.55 (m, 1H), 7.45 (ddd, J=27.97, 8.01, 1.79 Hz, 1H), 7.38 (m, 1H), 7.23-7.12(m, 3H), 6.56 (d, J=8.67 Hz, 1H), 4.80 (t, J=4.71 Hz, 1H), 4.15 (q, J=5.27 Hz, 1H), 3.53 (q, J=5.77 Hz, 2H), 3.16(d, J=5.27 Hz, 1H), 2.21 (s, 3H)

API-MS m/z 507.2([M+H]⁺, calcd 507.2)

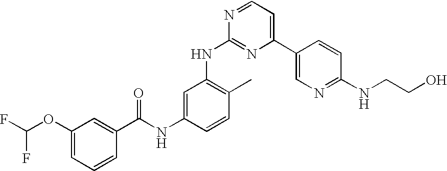

### EXAMPLE 17

### N-{3-[4-(6-Chloro-pyridin-3-yl)-pyrimidin-2-ylamino]-4-methyl-phenyl}-3-trifluoromethyl-benzamide

N*3*-[4-(6-Chloro-pyridin-3-yl)-pyrimidin-2-yl]-4,N*1*-dimethyl-benzene-1,3-diamine (1.34 g, 4.298 mmoles), 3-trifluoromethyl-benzoic acid (0.8575 g, 4.51 mmoles), BOP (2.28 g, 5.16 mmoles) are dissolved in 10 mL of DMF. Then DIEA (2.8 ml, 15.47 mmoles) is added to above mixture and the mixture is stirred at room temperature overnight. The dark reaction mixture is diluted with water. The precipitate is washed with water, saturated sodium carbonate, water and cold methanol. The resulting product is a yellow solid (1.66 g, yield 80%).

¹H NMR (DMSO, 300 MHz): δ (ppm) 10.44 (s, 1H), 9.13 (d, J=2.26 Hz, 1H), 9.05 (s,1H), 8.57-8.50 (m, 2H), 8.28 (d, J=11.68 Hz, 2H), 8.10 (d, J=1.89 Hz, 1H), 7.97 (d, 7.91 Hz, 1H), 7.79 (t, J=7.91 Hz, 1H), 7.66 (d, J=8.29 Hz, 1H), 7.51-7.42 (m, 2H), 7.24 (d, J=8.29 Hz, 1H), 2.23 (s, 3H)

API-MS m/z 484.37, 486.45 ([M+H]⁺, [(M+2)+H]⁺, calcd 483.12,486.12)

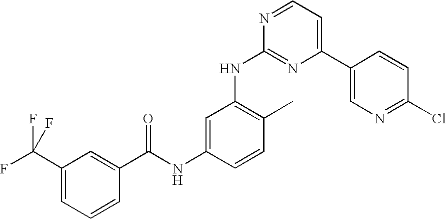

### EXAMPLE 18

### N-{3-[4-(6-Dimethylamino-pyridin-3-yl)-pyrimidin-2-ylamino]-4-methyl-phenyl}-3-trifluoromethyl-benzamide

N*3*-[4-(6-Dimethylamino-pyridin-3-yl)-pyrimidin-2-yl]-4-methyl-benzene-1,3-diamine (0.2 g, 0.6242 mmoles), 3-trifluoromethyl-benzoic acid (0.1246 g, 0.655 mmoles), BOP (0.331 g, 0.749 mmoles) are dissolved in 5 mL of DMF. DIEA (0.4 mL, 2.247 mmoles) is added and the mixture is stirred at room temperature overnight. The dark reaction mixture is diluted with water. The precipitate is chromatographed on silica gel with 80% ethyl acetate hexanes. The resulting product is a yellow solid (180mg, yield 61%).

¹H NMR (DMSO, 300 MHz): δ (ppm) 10.44 (s, 1H), 8.86 (d, J=2.26 Hz, 1H), 8.72 (s,1H), 8.34 (d, J=5.27 Hz, 1H), 8.28 (d, J=9.42 Hz, 2H), 8.21 (dd, J=9.04, 2.26 Hz, 1H), 8.09 (d, J=2.26 Hz, 1H), 7.96 (d, J=7.91 Hz, 1H), 7.78 (t, J=7.72 Hz, 1H), 7.48 (dd, J=8.10, 2.07 Hz, 1H), 7.28-7.19 (m, 2H), 6.68 (d, J=9.04 Hz, 1H), 3.09 (s, 6H), 2.23 (s, 3H)

API-MS m/z, 493.5 ([M+H]⁺, calcd 493.5)

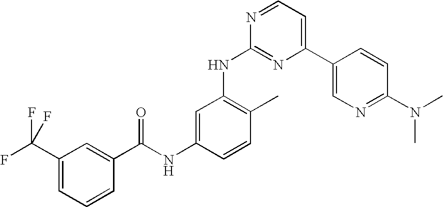

### EXAMPLE 19

### N-(3-{4-[6-(2-Methoxy-ethoxy)-pyridin-3-yl]-pyrimidin-2-ylamino}-4-methyl-phenyl)-3-trifluoromethyl-benzamide

NaH (90.9 mg, 2.27 mmoles, 60%) is added to the solution of 2-Methoxy-ethanol (78.6 mg, 1.033 mmoles) at room temperature. The reaction mixture is kept stirring at room temperature for 30 min before N-{3-[4-(6-Chloro-pyridin-3-yl)-pyrimidin-2-ylamino]-4-methyl-phenyl}-3-trifluoromethyl-benzamide (50 mg, 0.1033 mmoles) is added. The reaction mixture is further stirred at room temperature for another 4 hours. The reaction mixture is diluted with water and the precipitate filtered to obtain a white solid (64 mg, 100%).

¹H NMR (DMSO, 300 MHz): δ (ppm) 10.44 (s, 1H), 8.95-8.85 (m, 2H), 8.55-8.35 (m,2H), 8.30-8.24 (m, 2H), 8.10 (s, 1H), 7.97 (d, J=7.92 Hz, 1H), 7.79 (t, J=7.54 Hz, 1H), 7.45 (d, J=8.29Hz, 1H), 7.36 (d, J=4.90 Hz, 1H), 7.23 (d, J=8.29 Hz, 1H), 6.93 (d, J=8.67 Hz, 1H), 4.43 (t, J=4.52 Hz, 2H), 3.66 (t, J=4.52 Hz, 2H), 3.29 (s, 3H), 2.23 (s, 3H)

API-MS m/z 524.5 ([M+H]⁺, calcd 524.5

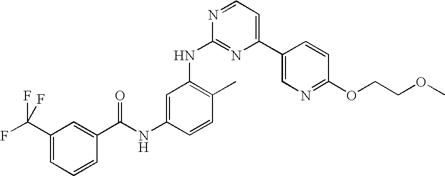

### EXAMPLE 20

### N-(4-Methyl-3-{4-[6-(4-methyl-piperazin-1-yl)-pyridin-3-yl]-pyrimidin-2-ylamino}-phenyl)-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) is stirred in 1 g of 1-Methyl-piperazine at 110° C. overnight. The reaction mixture is diluted with water and the yellow precipitate is collected by filtration. After drying under vacuum, a white solid is obtained (55 mg, yield 97.2%).

¹H NMR (DMSO, 300 MHz): δ (ppm) 10.44 (s, 1H), 8.95-8.85 (m, 2H), 8.55-8.35 (m,2H), 8.30-8.24 (m, 2H), 8.10 (s, 1H), 7.97 (d, J=7.92 Hz, 1H), 7.79 (t, J=7.54 Hz, 1H), 7.45 (d, J=8.29 Hz, 1H), 7.36 (d, J=4.90 Hz, 1H), 7.23 (d, J=8.29 Hz, 1H), 6.93 (d, J=8.67 Hz, 1H), 4.43 (t, J=4.52 Hz, 2H), 3.66 (t, J=4.52 Hz, 2H), 3.29 (s, 3H), 2.23 (s, 3H)

API-MS m/z 524.5 ([M+H]⁺, calcd 524.5

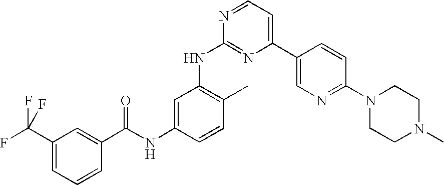

### EXAMPLE 21

### N-{4-Methyl-3-[4-(6-methylamino-pyridin-3-yl)-pyrimidin-2-ylamino]-phenyl}-3-trifluoromethyl-benzamide

N-{3-[4-(6-Chloro-pyridin-3-yl)-pyrimidin-2-ylamino]-4-methylphenyl}-3-trifluoromethyl-benzamide (50 mg, 0.103 mmoles) is stirred in 2 mL of methyl amine (2 M in THF) at 110° C. overnight. The reaction mixture is diluted with water and the yellow precipitate is collected by filtration. After drying under vacuum, a white solid is obtained (31 mg, yield 63%).

¹H NMR (DMSO, 300 MHz): δ (ppm) 10.44 (s, 1H), 8.78(d, J=2.26 Hz, 1H), 8.70(s, 1H), 8.35-8.24 (m, 3H), 8.15-8.05 (m, 2H), 7.97(d, J=1.87 Hz, 1H), 7.78 (t, J=7.72 Hz, 1H), 7.47 (dd, J=8.10, 2.07 Hz, 1H), 7.24-7.18 (m, 2H), 7.10-7.00 (m, 1H), 6.49 (d, J=8.67 Hz, 1H), 2.81 (d, J=4.52 Hz, 3H), 2.23 (s, 3H)

API-MS m/z 479.2 ([M+H]⁺, calcd 479.2

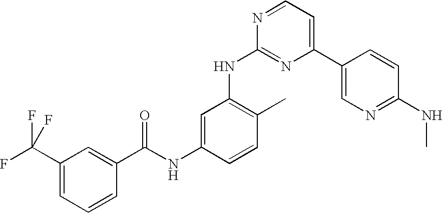

### EXAMPLE 22

### N-{3-[4-(6-Cyclopropylamino-pyridin-3-yl)-pyrimidin-2-ylamino]-4-methyl-phenyl}-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) is stirred in 1 mL of Cyclopropylamine at 110° C. overnight. The reaction mixture is diluted with water. The precipitate is collected by filtration and recrystallized in ether. After drying under vacuum, a white solid is obtained (6 mg, yield 11.5%).

¹H NMR (DMSO, 300 MHz): δ (ppm) 10.44 (s, 1H), 8.78(d, J=2.26 Hz, 1H), 8.70(s, 1H), 8.35-8.24 (m, 3H), 8.19 (dd, J=8.85, 2.45 Hz, 1H), 8.08(d, J=1.88 Hz, 1H), 7.96 (d, J=7.91 Hz, 1H), 7.78 (t, J=7.72 Hz, 1H), 7.47 (dd, J=8.10, 1.70 Hz, 1H), 7.33 (d, J=2.26 Hz, 1H), 7.50-7.40 (m, 2H), 6.63 (d, J=8.67 Hz, 1H), 2.60-2.53 (m, 1H), 2.23 (s, 3H), 0.76-0.68 (m, 2H), 0.48-0.40 (m, 2H)

API-MS m/z 505.3 ([M+H]⁺, calcd 505.2

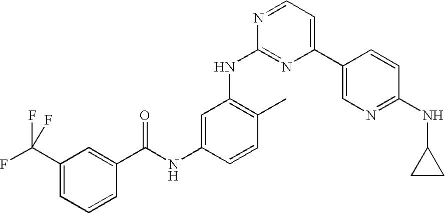

### EXAMPLE 23

### N-{3-[4-(6-Cyclopentylamino-pyridin-3-yl)-pyrimidin-2-ylamino]-4-methyl-phenyl}-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) is stirred in 1 ml of Cyclopentylamine at 110 C° overnight. The reaction mixture is diluted with water. The precipitate is collected by filtration and recrystallized from ether. After drying under vacuum, a white solid is obtained (27 mg, yield 49.2%).

¹H NMR (DMSO, 300 MHz): δ (ppm) 10.44 (s, 1H), 8.76(d, J=2.26 Hz, 1H), 8.67(s, 1H), 8.36-8.24 (m, 3H), 8.13-8.04 (m, 2H), 7.97 (d, J=7.91 Hz, 1H), 7.78 (t, J=7.72 Hz, 1H), 7.47 (dd, J=8.28, 1.88 Hz, 1H), 7.25-7.15 (m, 2H),7.11-7.04 (m, 1H), 6.48 (d, J=9.04 Hz, 1H), 4.21-4.05 (m, 1H), 2.23 (s, 3H), 2.00-1.85(m, 2H), 1.75-1.35 (m, 6H)

API-MS m/z 533.3 ([M+H]⁺, calcd 533.2

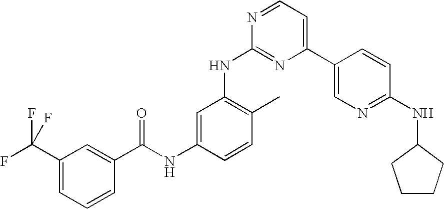

### EXAMPLE 24

### N-{4-Methyl-3-[4-(6-thiomorpholin-4-yl-pyridin-3-yl)-pyrimidin-2-ylamino]-phenyl}-3-trifluoromethyl-benzamide

N-{3-[4-(6-Chloro-Pyridin-3-yl)-pyrimidin-2-ylamino]-4-methyl-phenyl}-3-trifluoromethyl-benzamide (50 mg, 0.103 mmoles) is stirred in 1 mL of Thiomorpholine at 110° C. overnight. The reaction mixture is diluted with water and the precipitate is collected by filtration. After drying under vacuum, a white solid is obtained (50 mg, yield 87.9%).

¹H NMR (DMSO, 300 MHz): δ (ppm) 10.44 (s, 1H), 8.87 (d, J=2.26 Hz, 1H), 8.76 (s, 1H), 8.36 (d, J=5.27 Hz, 1H), 8.32-8.20 (m, 3H), 8.07 (d, J=1.88 Hz, 1H), 7.96 (d, J=7.16 Hz, 1H), 7.78 (t, J=7.91 Hz, 1H), 7.47 (dd, J=8.10, 2.07 Hz, 1H), 7.28-7.18 (m, 2H),7.11-7.04 (m, 1H), 6.91 (d, J=9.04 Hz, 1H), 4.25-3.94 (m, 4H), 2.65-2.56(m, 4H), 2.23 (s, 3H)

API-MS m/z 551.5 ([M+H]⁺, calcd 551.3

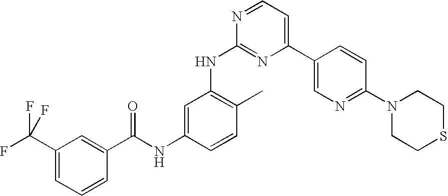

### EXAMPLE 25

### N-{4-Methyl-3-[4-(6-morpholin-4-yl-pyridin-3-yl)-pyrimidin-2-ylamino]-phenyl}-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) is stirred in 1 mL of morpholine at 110° C. overnight. The reaction mixture is diluted with water and the precipitate is collected by filtration. After drying under vacuum, a white solid is obtained (46 mg, yield 83.3%).

¹H NMR (DMSO, 300 MHz): δ (ppm) 10.44 (s, 1H), 8.89 (d, J=1.88 Hz, 1H), 8.77 (s, 1H), 8.36 (d, J=5.27 Hz, 1H), 8.32-8.20 (m, 3H), 8.08 (d, J=1.88 Hz, 1H), 7.96 (d, J=7.53 Hz, 1H), 7.78 (t, J=7.72 Hz, 1H), 7.48 (dd, J=8.29, 2.26 Hz, 1H), 7.28-7.18 (m, 2H), 6.90 (d, J=8.67 Hz, 1H), 3.75-3.65 (m, 4H), 3.60-3.5(m, 4H), 2.23 (s, 3H)

API-MS m/z 535.5 ([M+H]⁺, calcd 535.3

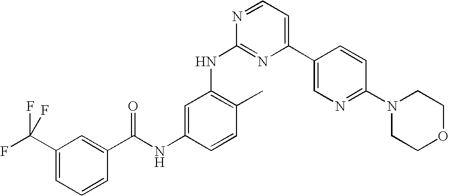

### EXAMPLE 26

### N-{3-[4-(4-Hydroxy-3,4,5,6-tetrahydro-2H-[1,2′]bipyridinyl-5′-yl)-pyrimidin-2-ylamino]-4-methyl-phenyl}-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) is stirred in 1 mL of Piperidin-4-ol at 110° C. overnight. The reaction mixture is diluted with water and the precipitate is collected by filtration. After drying under vacuum, a white solid is obtained (54 mg, yield 98.4%).

¹H NMR (DMSO, 300 MHz): δ (ppm) 10.44 (s, 1H), 8.86 (d, J=1.88 Hz, 1H), 8.73 (s, 1H), 8.43-8.18 (m, 4H), 8.10 (s, 1H), 7.96 (d, J=7.91 Hz, 1H), 7.78 (t, J=7.53 Hz, 1H), 7.48 (dd, J=8.47, 0.94 Hz, 1H), 7.28-7.18 (m, 2H), 6.90 (d, J=9.04 Hz, 1H), 4.75-4.69 (m, 1H), 4.15-4.00(m, 2H), 3.79-3.66 (m, 1H), 3.29-3.15 (m, 2H),2.23 (s, 3H), 1.84-1.71 (m, 2H), 1.42-1.20 (m, 2H);

API-MS m/z 549.5 ([M+H]⁺, calcd 549.3

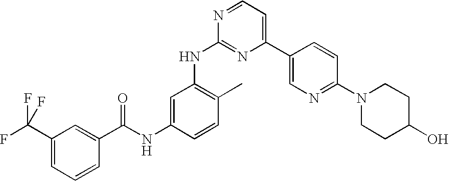

### EXAMPLE 27

### N-(3-{4-[6-(3-Diethylamino-propylamino)-pyridin-3-yl]-pyrimidin-2-ylamino}-4-methyl-phenyl)-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) is stirred in 1 ml of N*1*,N*1*-Diethyl-propane-1,3-diamine at 110° C. overnight. The reaction mixture is diluted with water and the precipitate is collected by filtration. After drying under vacuum, a white solid is obtained (80 mg, yield 100%).

¹H NMR (DMSO, 300 MHz): δ (ppm) 10.50 (s, 1H), 8.76 (d, J=1.88 Hz, 1H), 8.69 (s, 1H), 8.32-8.25 (m, 3H), 8.13-8.05 (m, 2H), 7.96 (d, J=7.53 Hz, 1H), 7.78 (t, J=8.10 Hz, 1H), 7.48 (dd, J=8.10, 1.70 Hz, 1H), 7.25-7.18 (m, 2H), 6.50 (d, J=9.04 Hz, 1H), 3.33-3.23 (m, 2H), 2.5-2.35(m, 6H), 2.23 (s, 3H), 1.03-0.85(m, 6H)

API-MS m/z 578.6 ([M+H]⁺, calcd 578.4

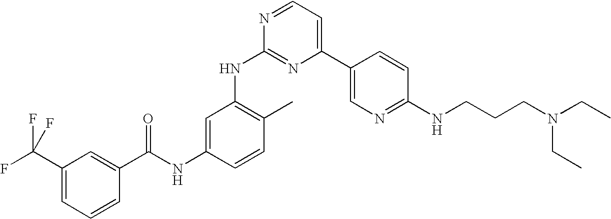

### EXAMPLE 28

### N-[4-Methyl-3-(4-{6-[(pyridin-4-ylmethyl)-amino]-pyridin-3-yl}-pyrimidin-2-ylamino)-phenyl]-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) is stirred in 1 mL of C-Pyridin-4-yl-methylamine at 110 C° overnight. The reaction mixture is diluted with water. The precipitate is collected by filtration and washed with ether. After drying under vacuum, a white solid is obtained (30 mg, yield 52.4%).

¹H NMR (DMSO, 300 MHz): δ (ppm) 10.42 (s, 1H), 8.73 (d, J=1.88 Hz, 1H), 8.70 (s, 1H), 8.46 (d, J=6.03 Hz, 2H), 8.32 (d, J=5.27 Hz, 1H), 8.31-8.23 (m, 2H), 8.13 (dd, J=8.67, 2.26 Hz, 1H), 8.05(d, J=1.88 Hz, 1H), 7.96 (d, J=7.91 Hz, 1H), 7.82-7.69 (m, 2H), 7.46 (dd, J=8.29, 2.26 Hz, 1H), 7.28 (d, J=5.65, 2H), 7.24-7.15 (m, 2H), 6.61 (d, J=8.67 Hz, 1H), 4.57 (d, J=5.65 Hz, 2H),2.23 (s, 3H)

API-MS m/z 556.5 ([M+H]⁺, calcd 556.5

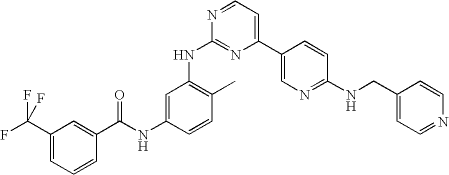

### EXAMPLE 29

### N-(3-{4-[6-(2-Methoxy-ethylamino)-pyridin-3-yl]-pyrimidin-2-ylamino}-4-methyl-phenyl)-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) is stirred in 1 mL of 2-Methoxy-ethylamine at 110° C. overnight. The reaction mixture is diluted with water and the precipitate is collected by filtration and washed with ether. After drying under vacuum, a yellow solid is obtained (65 mg, yield 66.9%).

¹H NMR (DMSO, 300 MHz): δ (ppm) 10.42 (s, 1H), 8.76 (d, J=2.26 Hz, 1H), 8.69 (s, 1H), 8.35-8.25 (m, 3H), 8.13-8.05 (m, 2H), 7.96 (d, J=7.96 Hz, 1H), 7.78 (t, J=7.72 Hz, 1H), 7.48 (dd, J=8.10, 2.07 Hz, 1H), 7.25-7.13 (m, 3H), 6.56 (d, J=9.04 Hz, 1H), 3.48-3.43 (m, 4H), 3.26(s, 3H),3.17 (d, J=5.27 Hz, 2H),2.23 (s, 3H)

API-MS m/z 523.5 ([M+H]⁺, calcd 523.5

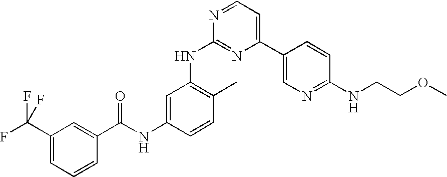

### EXAMPLE 30

### N-(3-{4-[6-(2-Hydroxy-propylsulfanyl)-pyridin-3-yl]-pyrimidin-2-ylamino}-4-methyl-phenyl)-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles) is stirred in 1 mL of 1-Mercapto-propan-2-ol in the presence of potassium carbonate at room temperature overnight. The reaction mixture is diluted with water. The precipitate is collected by filtration and washed with ether. After drying under vacuum, a solid was obtained (11 mg, yield 20%).

¹H NMR (DMSO, 300 MHz): δ (ppm) 10.44 (s, 1H), 9.12 (d, J=2.26 Hz, 1H), 8.94 (s, 1H), 8.47 (d, J=5.27 Hz, 1H), 8.35-8.25 (m, 3H), 8.08 (d, J=1.88 Hz, 1H), 7.96 (d, J=7.91 Hz, 1H), 7.78 (t, J=7.72 Hz, 1H), 7.48 (dd, J=8.28, 1.88 Hz, 1H), 7.45-7.35 (m, 2H), 7.23 (d, J=8.28 Hz, 1H), 4.99 (br, 1H), 3.90-3.80(m, 1H),3.22 (d, J=6.02 Hz, 2H),2.23 (s, 3H), 1.14 (d, J=6.02 Hz, 3H)

API-MS m/z 540.4 ([M+H]⁺, calcd 540.5

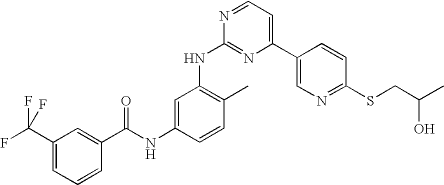

### EXAMPLE 31

### N-(4-Methyl-3-{4-[6-(pyridin-3-yloxy)-pyridin-3-yl]-pyrimidin-2-ylamino}-phenyl)-3-trifluoromethyl-benzamide

N-{3-[4-(6-Chloro-pyridin-3-yl)-pyrimidin-2-ylamino]-4-methyl-phenyl}-3-trifluoromethyl-benzamide (50 mg, 0.103 mmoles), Pyridin-3-ol (98.7 mg, 1.03 mmoles), 18-crown-6 (20 mg), KOH (56 mg) is stirred in 1 mL of toluene at 110° C. overnight. The reaction mixture is diluted with water. The precipitate is collected by filtration and further purified by prep-TLC (100% ethyl acetate). After drying under vacuum, a white solid is obtained (11.3 mg, yield 26.2%).

¹H NMR (DMSO, 300 MHz): δ (ppm) 10.44 (s, 1H), 8.92(s, 1H), 8.86 (d, J=2.26 Hz, 1H), 8.59 (dd, J=8.67, 2.64 Hz, 1H), 8.53-8.45 (m, 3H), 8.30-8.22(m, 2H), 8.08 (d, J=1.88 Hz, 1H), 7.96 (d, J=7.91 Hz, 1H), 7.78 (t, J=7.72 Hz, 1H), 7.69 (dq, J=8.38, 1.38 Hz, 1H), 7.53-7.44 (m, 2H), 7.37 (d, J=5.27 Hz, 1H), 7.27-7.20 (m, 2H), 2.23 (s, 3H)

API-MS m/z 543.3 ([M+H]⁺, calcd 543.3

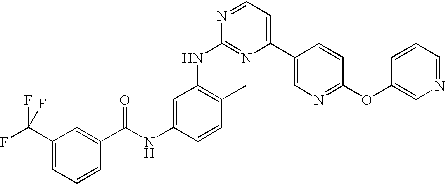

### EXAMPLE 32

### N-(4-Methyl-3-{4-[6-(1-methyl-piperidin-4-yloxy)-pyridin-3-yl]-pyrimidin-2-ylamino}-phenyl)-3-trifluoromethyl-benzamide

NaH (90.9 mg, 2.27 mmoles, 60%) is added to the solution of 1-Methyl-piperidin-4-ol (119 mg, 1.033 mmoles) in 2 mL of DMF at room temperature. The reaction mixture is stirred at room temperature for 30 min before N-{3-[4-(6-Chloro-pyridin-3-yl)-pyrimidin-2-ylamino]-4-methyl-phenyl}-3-trifluoromethyl-benzamide (50 mg, 0.1033 mmoles) is added. The reaction mixture is further stirred at room temperature overnight. The reaction mixture is diluted with water and the precipitate is obtained by filtration and further purified by PREP-TLC (60% Ethyl acetate/hexanes). After drying under vacuum, a solid product is obtained (50 mg, yield 86.3%).

¹H NMR (DMSO, 300 MHz): δ (ppm) 10.44 (s, 1H), 8.90 (d, J=2.26 Hz, 1H), 8.88(s, 1H), 8.46 (d, J=5.27 Hz, 1H), 8.39 (dd, J=8.85, 2.45 Hz, 1H), 8.48-8.24 (m, 2H), 8.11 (d, J=1.88 Hz, 1H), 7.96 (d, J=7.91 Hz, 1H), 7.78 (t, J=7.72 Hz, 1H), 7.48 (dd, J=8.28, 1.88 Hz, 1H), 7.34 (d, J=5.27 Hz, 1H), 7.23 (d, J=8.29 Hz, 1H), 6.87 (d, J=8.67 Hz, 1H), 5.1-5.0 (m, 1H), 2.71-2.59 (m, 2H), 2.23 (s, 3H), 2.21 (s, 3H), 2.3-2.1 (m, 2H), 2.02-1.91 (m, 2H), 1.75-1.59 (m, 2H)

API-MS m/z 563.4 ([M+H]⁺, calcd 563.5

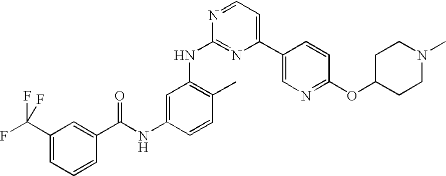

### EXAMPLE 33

### N-[3-([4,5′]Bipyrimidinyl-2-ylamino)-4-methyl-phenyl]-3-trifluoromethyl-benzamide

A mixture of N*3*-[4,5′]Bipyrimidinyl-2-yl-4-methyl-benzene-1,3-diamine (0.23 g, 0.83 mmol), 3-Trifluoromethyl-benzoic acid (0.16 g, 0.87 mmol), BOP (0.44 g, 1.00 mmol) and DIEA (0.5 mL, 3.00 mmol) is stirred in 3.0 mL of DMF at room temperature overnight. 40 mL of water is added to the precipitate. The resulting suspension is allowed to stir at room temperature for I hour. It is then filtered through a Buchner funnel, triturated twice with 5.0 mL of CH₃OH and dried. A brown solid is obtained.

The solution is purified by flash chromatography using 1:5 EtOAc:Hexane→1:1 EtOAc:Hexane. The solvent is removed under vacuum. 0.14 g of N-[3-([4,5′]Bipyrimidinyl-2-ylamino)-4-methyl-phenyl]-3-trifluoromethyl-benzamide is obtained as a yellow solid.

¹H NMR (DMSO, 500 MHz): δ (ppm) 10.50 (s, 1H), 9.40 (s, 1H), 9.18 (s, 1H), 9.05 (d,1H, J=3.78 Hz), 8.71 (d, 1H, J=4.16 Hz), 8.46-8.27 (m, 3H), 8.17 (s, 1H), 8.00 (d, 1H, J=7.14Hz), 7.83 (dd, 1H, J=6.7 Hz), 7.75(d, 1H, J=3.96 Hz), 7.64-7.43 (m, 2H), 7.36-7.20 (m, 1H), 2.29 (s, 3H).

API-MS m/z 451.2 ([M+H]⁺, calcd 451.1)

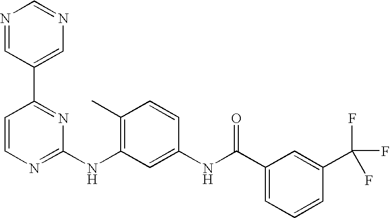

### EXAMPLE 34

### 3-Fluoro-N-[4-methyl-3-(4-pyrazin-2-yl-pyrimidin-2-ylamino)-phenyl]-5-trifluoromethyl-benzamide

A mixture of 4-Methyl-N*3*-(4-pyrazin-2-yl-pyrimidin-2-yl)-benzene-1,3-diamine (0.55 g, 1.98 mmol), 3-Fluoro-5-trifluoromethyl-benzoic acid (0.43 g, 2.08 mmol), BOP (1.05 g, 2.38 mmol) and DIEA (1.25 mL, 7.20 mmol) is stirred in 9.0 mL of DMF at room temperature overnight. 25 mL of water is added to the precipitate. The resulting suspension is allowed to stir at room temperature for one hour. It is then filtered through a Buchner funnel, air dried and triturated with 10.0 mL of CH₃OH for 1 hour and dried. 0.80 9 of 3-Fluoro-N-[4-methyl-3-(4-pyrazin-2-yl-pyrimidin-2-ylamino)-phenyl]-5-trifluoromethyl-benzamide is obtained as a pale yellow solid.

¹H NMR (DMSO, 500 MHz): δ (ppm) 10.49 (s, 1H), 9.49 (s, 1H), 9.10 (s, 1H), 8.79 (s,2H), 8.62 (d, 2H, J=4.81 Hz), 8.10-8.20 (m, 3H), 7.96 (d, 1H, J=8.66 Hz), 7.63 (d, 1H,J=4.78 Hz), 7.48 (dd, 1H,J₁=8.46 Hz, J₂=2.05 Hz), 7.26 (d, 1H,J=8.09 Hz), 2.26 (s,3H).

API-MS m/z 469.1 ([M+H]⁺, calcd 469.1)

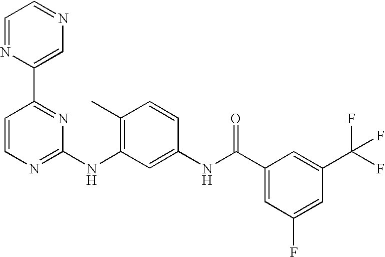

### Biological Activity

Active B-Raf, C-Raf, and V599E B-Raf proteins of human sequence are purified from insect cells using the baculoviral expression system. Raf inhibition is tested in 96-well microplates coated with IκB-α and blocked with Superblock. The phosphorylation of IκB-α at Serine 36 is detected using a phospho-IκB-α specific antibody (Cell Signaling #9246), an anti-mouse IgG alkaline phosphatase conjugated secondary antibody (Pierce #31320), and an alkaline phosphatase substrate, ATTOPHOS (Promega, # S101).

Each of the compounds of Examples 1-34 inhibits wild-type C-RAF at an IC₅₀ of from 0.01 to 0.7 micromoles/liter and/or wild-type B-RAF at an IC₅₀ of from 0.04 to 1.5 micromoles/liter and/or mutant B-RAF (V599E) at an IC₅₀ of from 0.006 to 1.6 micromoles/liter.

### EXAMPLE D1

Detection of T1796A Mutation in the Human B Raf Gene

| | |
|---|---|
| Detection Primer: | GATTTTGGTCTAGCTACAGA |
| Second Primer: | GACTTTCTAGTAACTCAGCAG |

Genomic DNA is isolated from human cells from a melanoma cell line using a GENELUTE mammalian genomic DNA kit (Sigma cat # G1N 350). PCR reactions are carried out on a PCR machine (MJ Research, Model PTC100) in a total volume of 50 microL using the PCR Core kit by Roche (cat #1578 553). The PCR reaction mixture contains 5 microL of 10× reaction buffer, 1 microL of 10 mM dNTPs, 100-1000 ng of template DNA, 0.5 microL Taq polymerase (2.5-5 units), 1 microL of a 31 uM stock of each primer.

The PCR conditions are as follows:

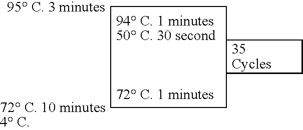

After amplification, 8 microLs of the PCR reaction mixture is mixed with 2 microLs of nucleic acid sample loading buffer [BioRad cat #161-0767]. The 10 microL sample is loaded onto a 1.5% agarose [GIBCO-BRL cat #15510-027] gel that contains 0.3 ug/mI of ethidium bromide [Pierce cat #17898]. Molecular weight standards [100 bp DNA ladder from Invitrogen cat #10380-012] are loaded in an adjacent lane. The DNA is separated by electrophoresis in TAE buffer (0.04 M Tris-acetate, 0.01 M EDTA. 0.02M glacial acetic acid pH8.4)[Roche cat #1666690]. Electrophoresis conditions are 120 volts for 30-40 minutes. After separation, the gel is exposed to UV light and a picture taken on an Alphalmager2000 documentation system.

Generally, two bands are detected in the gel. The faster migrating band runs ahead of the 100 bp marker and represents the primers. The DNA that results from the T1796A mutant specific PCR amplification has a predicted size of 152 bp and migrates between the 100 bp standard and the 200 bp standard as predicted. The PCR amplification product is confirmed by sequencing. The presence of the PCR amplification product demonstrates that the T1796A mutation is present in the template DNA. The absence of the PCR amplification product is evidence that the mutation is absent in the tissue sample.

Other B-RAF mutations are detected by this method utilizing the detection primer and second primer indicated for the mutation in the following tables:

| | | |
|---|---|---|
| SEQ | Detection primer | |
| ID | oligonucleotide | B-RAF |
| NO: | segment (5′→3′) | mutation |

| | | |
|---|---|---|
| 1 | ACAGTGGGACAAAGAATTGA | G1388A |
| 2 | ACAGTGGGACAAAGAATTGT | G1388T |
| 3 | GGACAAAGAATTGGATCTGC | G1394C |
| 4 | GGACAAAGAATTGGATCTGA | G1394A |
| 5 | GGACAAAGAATTGGATCTGT | G1394T |
| 6 | ATTGGATCTGGATCATTTGC | G1403C |
| 7 | ATTGGATCTGGATCATTTGA | G1403A |
| 8 | GAGTAATAATATATTTCTTCATA | G1753A |
| 9 | CAGTAAAAATAGGTGATTTG | T1782G |
| 10 | CAGTAAAAATAGGTGATTTTC | G1783C |
| 11 | GTAAAAATAGGTGATTTTGGTG | C1786G |
| 12 | GTAAAAATAGGTGATTTTGGTCG | T1787G |
| 13 | GATTTTGGTCTAGCTACAGA | T1796A |
| 14 | GATTTTGGTCTAGCTACAGAT | TG1796-97AT |

| SEQ | Second Primer | |
|---|---|---|
| ID | oligonucleotide | B-RAF |
| NO: | segment (5′→3′) | mutation |
| 15 | TGTCACCACATTACATACTTACC | G1388A |
| 16 | TGTCACCACATTACATACTTACC | G1388T |
| 17 | TGTCACCACATTACATACTTACC | G1394C |
| 18 | TGTCACCACATTACATACTTACC | G1394A |
| 19 | TGTCACCACATTACATACTTACC | G1394T |
| 20 | TGTCACCACATTACATACTTACC | G1403C |
| 21 | TGTCACCACATTACATACTTACC | G1403A |
| 22 | GACTTTCTAGTAACTCAGCAG | G1753A |
| 23 | GACTTTCTAGTAACTCAGCAG | T1782G |
| 24 | GACTTTCTAGTAACTCAGCAG | G1783C |
| 25 | GACTTTCTAGTAACTCAGCAG | C1786G |
| 26 | GACTTTCTAGTAACTCAGCAG | T1787G |
| 27 | GACTTTCTAGTAACTCAGCAG | T1796A |
| 28 | GACTTTCTAGTAACTCAGCAG | TG1796-97AT |

1. A compound of the formula (I) 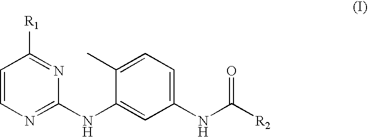
wherein
R₁ is a phenyl radical or a heteroaryl radical; and
R₂ is a phenyl radical;
or an N-oxide or a pharmaceutically acceptable salt thereof.

2. A compound of formula I wherein R₁ is selected from a phenyl radical, a thiazolyl radical, a pyrazinyl radical, a pyrimidinyl radical or a pyridyl radical.

3. The compound of claim 2 wherein R₂ is phenyl that is substituted in at least the 3-position by halogen, mono- or di-lower alkyl-substituted amino; lower alkyl; halo-lower alkyl; lower alkoxy; halo-lower alkoxy; lower alkylthio; or halo-lower alkylthio.

4. The compound of claim 3 wherein R₂ is phenyl that is substituted in at least the 3-position by fluorine, halo-lower alkyl, halo-lower alkoxy, or halo-lower alkylthio.

5. The compound of claim 1 wherein R₁ is a phenyl, 2-thiazolyl, 2-pyrazinyl, 5-pyrimidinyl or 3-pyridyl radical.

6. The compound of claim 5 wherein R₂ is phenyl that is substituted in at least the 3-position by fluorine, halo-lower alkyl, halo-lower alkoxy, or halo-lower alkylthio.

7. The compound of claim 1 of formula II 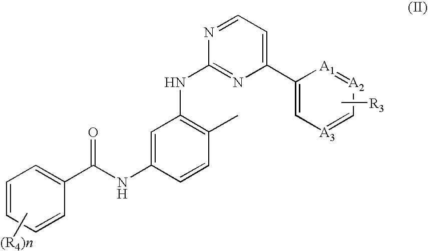
wherein
n is 0, 1 or 2;
A₁, A₂ and A₃ are C, or A₁ and A₂ are C and A₃ is N, or A₁ and A₃ are N and A₂ is C, or A₁ is C and A₂ and A₃ are N;
R₃ is —NR₅R₆, halogen, —O—R₈, —S—R₈, or lower alkyl which is unsubstituted or substituted by halogen, hydroxy, lower alkoxy, —NR₇R₈, or a heteroaryl or heterocyclic radical attached at a ring carbon;
R₄ is amino, mono- or di-lower alkyl-substituted amino, wherein the alkyl groups are unsubstituted or substituted by halogen or lower alkoxy; halogen, lower alkyl, halo-lower alkyl, lower alkoxy, halo-lower alkoxy, hydroxy, lower alkanoyl, carbamoyl, N-mono- or N,N-di-substituted carbamoyl, mercapto, lower alkylthio or halo-lower alkylthio;
R₅, R₆, R₇ and R₈ are independently hydrogen, a heteroaryl or heterocyclic radical attached at a ring carbon, lower alkyl, C₃-C₈cycloalkyl, C₃-C₈cycloalkyl-lower alkylene, lower alkyl which is substituted by hydroxy, lower alkoxy, a heteroaryl radical, a heterocyclic radical, amino, mono- or di-lower alkyl amino or R₅ and R₆ or R₇ and R₈ together with the nitrogen form a heteroaromatic or heterocyclic radical;
R₈ is a heterocyclic radical, a heteroaromatic radical, heteroaryl-lower-alkylene, heterocyclic-lower-alkylene, lower alkyl or lower alkyl which is substituted by hydroxy, lower alkoxy or —NR₇R₈;
or an N-oxide or a pharmaceutically acceptable salt thereof.

8. The compound of claim 7 wherein R₂ is phenyl that is substituted in at least the 3-position by halogen, mono- or di-lower alkyl-substituted amino; lower alkyl; halo-lower alkyl; lower alkoxy; halo-lower alkoxy; lower alkylthio; or halo-lower alkylthio.

9. The compound of claim 1 of formula (III) 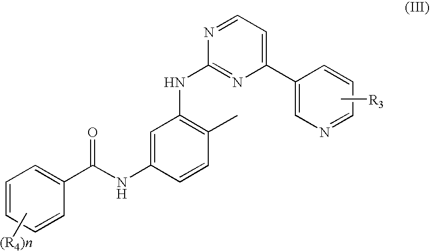
wherein
n is 0, 1 or 2;
R₃ is —NR₅R₆, halogen, —O—R₈, —S—R₈, or lower alkyl which is unsubstituted or substituted by halogen, hydroxy, lower alkoxy, —NR₇R₈, or a heteroaryl or heterocyclic radical attached at a ring carbon;
R₄ is amino, mono- or di-lower alkyl-substituted amino, wherein the alkyl groups are unsubstituted or substituted by halogen or lower alkoxy; halogen, lower alkyl, halo-lower alkyl, lower alkoxy, halo-lower alkoxy, hydroxy, lower alkanoyl, carbamoyl, N-mono- or N,N-di-substituted carbamoyl, mercapto, lower alkylthio or halo-lower alkylthio;
R₅, R₆, R₇ and R₈ are independently hydrogen, a heteroaryl or heterocyclic radical attached at a ring carbon, lower alkyl, C₃-C₈cycloalkyl, C₃-C₈cycloalkyl-lower alkylene, lower alkyl which is substituted by hydroxy, lower alkoxy, a heteroaryl radical, a heterocyclic radical, amino, mono- or di-lower alkyl amino or R₅ and R₆ or R₇ and R₈ together with the nitrogen form a heteroaromatic or heterocyclic radical;
R₈ is a heterocyclic radical, a heteroaromatic radical, heteroaryl-lower-alkylene, heterocyclic-lower-alkylene, lower alkyl or lower alkyl which is substituted by hydroxy, lower alkoxy or —NR₇R₈;
or an N-oxide or a pharmaceutically acceptable salt thereof.

10. The compound of claim 9 wherein R₄ is halogen, mono- or di-lower alkyl-substituted amino; lower alkyl; halo-lower alkyl; lower alkoxy; halo-lower alkoxy; lower alkylthio; or halo-lower alkylthio.

11. The compound of claim 10 wherein R₄ is phenyl halo-lower alkyl, halo-lower alkoxy or halo-lower alkylthio.

12. The compound of claim 9 wherein R₄ is trifluoromethyl.

13. The compound of claim 9 wherein R₃ is —NR₅R₆ and one of R₅ and R₆ is lower alkyl substituted by —NR₇R₈ and R₇ and R₈ together with the nitrogen form a heteroaromatic or heterocyclic radical.

14. The compound of claim 13 wherein the heteroaromatic or heterocyclic radical is selected from morphilino, thiomorphilino, piperazinyl, piperidinyl, and pyridyl.

15. The A compound of claim 9 wherein —NR₅R₆ is a heteroaryl or heterocyclic radical.

16. The compound of claim 15 wherein —NR₅R₆ is a heteroaryl or heterocyclic radical selected from piperazinyl, 4-methylpiperazinyl, piperidinyl, 4-hydroxypiperidinyl, morphilino and thiomorphilino.

17. The compound of claim 9 wherein R₈ is lower alkyl, lower alkyl substituted by hydroxy or lower alkoxy, or a heteroaryl or heterocyclic radical.

18. The compound of claim 9 of formula (IIIa)

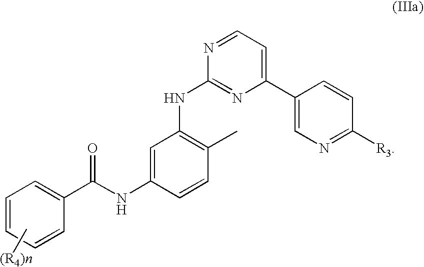

19. The compound of claim 9 of formula IIIb

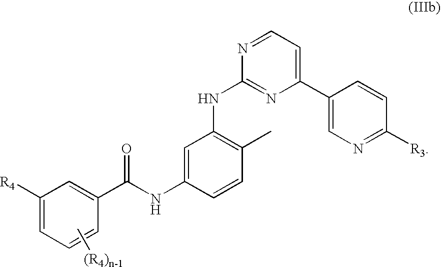

20. The compound of claim 7 of formula IV 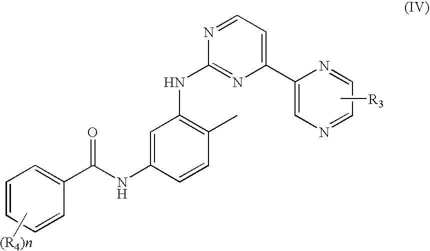
wherein
n is 0, 1 or 2;
R₃ is hydrogen, —NR₅R₆, halogen, —O—R₈, —S—R₈, or lower alkyl which is unsubstituted or substituted by halogen, hydroxy, lower alkoxy, —NR₇R₈, or a heteroaryl or heterocyclic radical attached at a ring carbon;
R₄ is amino, mono- or di-lower alkyl-substituted amino, wherein the alkyl groups are unsubstituted or substituted by halogen or lower alkoxy; halogen, lower alkyl, halo-lower alkyl, lower alkoxy, halo-lower alkoxy, hydroxy, lower alkanoyl, carbamoyl, N-mono- or N,N-di-substituted carbamoyl, mercapto, lower alkylthio or halo-lower alkylthio;
R₅, R₆, R₇ and R₈ are independently hydrogen, a heteroaryl or heterocyclic radical attached at a ring carbon, lower alkyl, C₃-C₈cycloalkyl, C₃-C₈cycloalkyl-lower alkylene, lower alkyl which is substituted by hydroxy, lower alkoxy, a heteroaryl radical, a heterocyclic radical, amino, mono- or di-lower alkyl amino or R₅ and R₆ or R₇ and R₈ together with the nitrogen form a heteroaromatic or heterocyclic radical;
R₈ is a heterocyclic radical, a heteroaromatic radical, heteroaryl-lower-alkylene, heterocyclic-lower-alkylene, lower alkyl or lower alkyl which is substituted by hydroxy, lower alkoxy or —NR₇R₈;
or a pharmaceutically acceptable salt thereof.

21. The compound of claim 20 wherein R₄ is halogen, mono- or di-lower alkyl-substituted amino; lower alkyl; halo-lower alkyl; lower alkoxy; halo-lower alkoxy; lower alkylthio; or halo-lower alkylthio.

22. The compound of claim 21 wherein at least one R4 substituent is in the meta position relative to the carbonyl.

23. The compound of claim 7 of the formula (V) 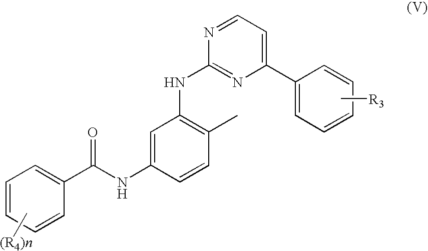
wherein
n is 0, 1 or2;
R₃ is —NR₅R₆, halogen, —O—R₈, —S—R₈, or lower alkyl which is unsubstituted or substituted by halogen, hydroxy, lower alkoxy, —NR₇R₈, or a heteroaryl or heterocyclic radical attached at a ring carbon;
R₄ is amino, mono- or di-lower alkyl-substituted amino, wherein the alkyl groups are unsubstituted or substituted by halogen or lower alkoxy; halogen, lower alkyl, halo-lower alkyl, lower alkoxy, halo-lower alkoxy, hydroxy, lower alkanoyl, carbamoyl, N-mono- or N,N-di-substituted carbamoyl, mercapto, lower alkylthio or halo-lower alkylthio;
R₅, R₆, R₇ and R₈ are independently hydrogen, a heteroaryl or heterocyclic radical attached at a ring carbon, lower alkyl, C₃-C₈cycloalkyl, C₃-C₈cycloalkyl-lower alkylene, lower alkyl which is substituted by hydroxy, lower alkoxy, a heteroaryl radical, a heterocyclic radical, amino, mono- or di-lower alkyl amino or R₅ and R₆ or R₇ and R₈ together with the nitrogen form a heteroaromatic or heterocyclic radical;
R₈ is a heterocyclic radical, a heteroaromatic radical, heteroaryl-lower-alkylene, heterocyclic-lower-alkylene, lower alkyl or lower alkyl which is substituted by hydroxy, lower alkoxy or —NR₇R₈;
or a pharmaceutically acceptable salt thereof.

24. The compound of claim 23 wherein R₄ is halogen, mono- or di-lower alkyl-substituted amino; lower alkyl; halo-lower alkyl; lower alkoxy; halo-lower alkoxy; lower alkylthio; or halo-lower alkylthio.

25. The compound of claim 24 wherein at least one R4 substituent is in the meta position relative to the carbonyl.

26. A method of treating a patient having a disease characterized by excessive signaling through the MAP kinase signaling pathway, which comprises administering to the patient an effective RAF kinase inhibiting amount of a compound of formula (I) according to claim 1.

27. A method of treating a patient having a disease characterized by excessive signaling through the MAP kinase signaling pathway, which comprises administering to the patient an effective RAF kinase inhibiting amount of a compound of formula (II) according to claim 7.

28. A method of treating a patient having a disease characterized by excessive signaling through the MAP kinase signaling pathway, which comprises administering to the patient an effective RAF kinase inhibiting amount of a compound of formula (III) according to claim 9.

28. A method of treating a patient having a disease characterized by excessive signaling through the MAP kinase signaling pathway, which comprises administering to the patient an effective RAF kinase inhibiting amount of a compound of formula (IIIb) according to claim 19.

29. A method of treating a patient having a disease characterized by excessive signaling through the MAP kinase signaling pathway, which comprises administering to the patient an effective RAF kinase inhibiting amount of a compound of formula (IV) according to claim 20.

30. A method of treating a patient having a disease characterized by excessive signaling through the MAP kinase signaling pathway, which comprises administering to the patient an effective RAF kinase inhibiting amount of a compound of formula (V) according to claim 23.

31. A process for the preparation of a compound of the formula (I), 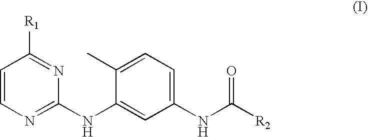
wherein
R₁ is a phenyl radical or a heteroaryl radical; and
R₂ is a phenyl radical;
or an N-oxide or a pharmaceutically acceptable salt thereof;
which process comprises preparing a compound of formula VIII by reacting a compound of formula VI with a compound of formula VII according to the following scheme

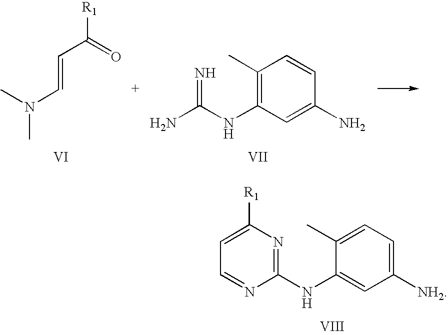

32. A compound of formula VII

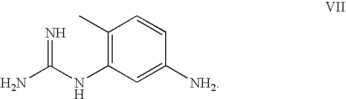

33. A compound of formula VIII 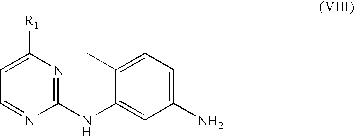
wherein R₁ is a phenyl radical or a heteroaryl radical.

34. A compound of the formula [chemical structure]


SEQUENCE LISTING (16 sequences)

SEQ ID NO: 1 - DNA, 20 bp, Homo sapiens
acagtgggacaaagaattga

SEQ ID NO: 2 - DNA, 20 bp, Homo sapiens
acagtgggacaaagaattgt

SEQ ID NO: 3 - DNA, 20 bp, Homo sapiens
ggacaaagaattggatctgc

SEQ ID NO: 4 - DNA, 20 bp, Homo sapiens
ggacaaagaattggatctga

SEQ ID NO: 5 - DNA, 20 bp, Homo sapiens
ggacaaagaattggatctgt

SEQ ID NO: 6 - DNA, 20 bp, Homo sapiens
attggatctggatcatttgc

SEQ ID NO: 7 - DNA, 20 bp, Homo sapiens
attggatctggatcatttga

SEQ ID NO: 8 - DNA, 23 bp, Homo sapiens
gagtaataatatatttcttcata

SEQ ID NO: 9 - DNA, 20 bp, Homo sapiens
cagtaaaaataggtgatttg

SEQ ID NO: 10 - DNA, 21 bp, Homo sapiens
cagtaaaaataggtgattttc

SEQ ID NO: 11 - DNA, 22 bp, Homo sapiens
gtaaaaataggtgattttggtg

SEQ ID NO: 12 - DNA, 23 bp, Homo sapiens
gtaaaaataggtgattttggtcg

SEQ ID NO: 13 - DNA, 20 bp, Homo sapiens
gattttggtctagctacaga

SEQ ID NO: 14 - DNA, 21 bp, Homo sapiens
gattttggtctagctacagat

SEQ ID NO: 15 - DNA, 20 bp, Homo sapiens
gattttggtctagctacaga

SEQ ID NO: 16 - DNA, 21 bp, Homo sapiens
gactttctagtaactcagcag